(19) United States
(12) Patent Application Publication
(10) Pub. No.: US 2006/0079461 A1
(43) Pub. Date: Apr. 13, 2006

(54) Treatment of multiple myeloma by inhibition of p38 MAP kinase

(75) Inventors: Richard Brewer, Santa Cruz, CA (US); Linda S. Higgins, Palo Alto, CA (US)

Correspondence Address: James J. Mullen III, Ph.D;MORRISON & FOERSTER LLP, Suite 100, 12531 High Bluff Drive, San Diego, CA, 92130-2040, US

(73) Assignee: Scios, Inc., Fremont, CA (US)

(21) Appl. No.: 11/207,542

(22) Filed: Aug. 19, 2005

Related U.S. Application Data
(63) Continuation-in-part of application No. 11/024,261, filed on Dec. 27, 2004.
(60) Provisional application No. 60/532,957, filed on Dec. 24, 2003.
(60) Provisional application No. 60/633,980, filed on Dec. 6, 2004.

(51) Int. Cl. A61K 38/06; A61K 38/05; A61K 31/454; A61K 31/445

(52) U.S. Cl. 514/18; 514/323; 514/319; 514/171

(57) ABSTRACT

The present invention provides a method to treat multiple myeloma by the administration of one or more p38 MAP kinase inhibitor(s).

### RELATED APPLICATIONS

This application is a continuation-in-part of co-pending U.S. patent application Ser. No. 11/024,261, filed on Dec. 27, 2004, which claims the benefit of priority of U.S. Provisional Patent Application No. 60/532,957, filed Dec. 24, 2003 and U.S. Provisional Patent Application No. 60/633,980, filed Dec. 6, 2004, all of which are hereby incorporated by reference in their entirety.

### FIELD OF THE INVENTION

The present invention provides a method to treat multiple myeloma using p38 MAP kinase inhibitors either alone or in combination with other chemotherapeutic compounds.

### BACKGROUND OF THE INVENTION

There are approximately 45,000 people in the United States living with multiple myeloma and an estimated 14,600 new cases of multiple myeloma are diagnosed each year. New cases of multiple myeloma thus represent twenty percent of blood cancers and one percent of all types of cancer. There is an annual incidence of multiple myeloma of approximately 4 in 100,000 in the United States. The prognosis for individuals diagnosed with multiple myeloma varies. The median survival with conventional therapies like dexamethasone, melphalan, prednisone, and bisphosphates, is about 2.5 to 3 years. Individuals treated with high dose chemotherapy and bone marrow transplant show a 5-year survival rate of greater than 50%. Although the cause of multiple myeloma is not known, risk factors for developing multiple myeloma include exposure to atomic radiation, petroleum products, pesticides, solvents, heavy metals and airborne particles.

Multiple myeloma (MM) is a neoplastic disease involving malignant plasma cells. These malignant plasma cells accumulate in bone marrow and typically produce monoclonal IgG or IgA molecules. Individuals suffering from multiple myeloma often experience anemia, osteolytic lesions, renal failure, hypercalcemia, and recurrent bacterial infections. Individuals with multiple myeloma frequently present increased monoclonal plasma cells in their bone marrow and serum or urinary monoclonal protein. For a general review of MM, see Bataille & Harousseau, JAMA (1997) 336(23):1657-1664.

Typically, a small number of long-lived plasma cells in the bone marrow produce most of the IgG and IgA molecules found in blood serum. These plasma cells are well differentiated and do not divide. Phenotypically, plasma cells are CD38^{bright}, syndecan-1^{bright}, CD19⁺, and CD56^{weak/-}. Precursors to the plasma cells are plasmablasts that migrate from the lymph nodes after antigen stimulation to the bone marrow. Once arriving in a germinal center, these stimulated cells switch from immature IgM production to IgG or IgA. After the stimulated cells enter the bone marrow, they stop dividing and differentiate into plasma cells. Plasma cells usually undergo apoptosis and die after several weeks or months.

Myeloma cells, in contrast to the normal plasma cells, display a phenotype reminiscent of the immature plasmablast. The myeloma cells usually display CD38, syndecan-1, CD19, and CD56^{bright}, and produce low amounts of immunoglobulins. Typically, the myeloma cells are aneuploid (hypoploid, but more often hyperploid) and their chromosomes have numerous structural abnormalities. Abnormalities are frequently apparent on chromosomes 13 (13q⁻) and 14 (14q⁺). The phenotypic characteristics of cellular immaturity and the 13q⁻ and 14q⁺ abnormalities correlate with resistance to treatment and to short survival characteristics of an aggressive disease state.

The myeloma cells adhere to and activate bone marrow stromal cells (BMSC) and are long-lived. Myeloma cells are dependent on interleukin-6 (IL-6), which is produced in copious quantities by BMSC. Interleukin-6 promotes MM cell growth. Hideshima, et al., Blood (2003) 101(2):703-705. Other cytokines are thought to be involved in the growth, survival, migration and adherence of MM cells and the development of osteolytic lesions. For example, vascular endothelial growth factor (VEGF) induces MM cell migration. Id. Adherence of MM cells to bone marrow stromal (BMSC) cells up-regulates IL-6 and VEGF secretion from both MM and BMSC cells.

The p38 mitogen-activated protein kinase (MAPK), which is a member of the MAPK family of kinases that is activated by cytokines and growth factors, may play a role in the multiple myeloma disease state. The exact role of p38 in MM, however, is unknown. One recent study demonstrated that a specific inhibitor of p38 MAPK inhibited IL-6 and vascular endothelial growth factor (VEGF) secretion in bone marrow stromal cells (BMSCs) without affecting the viability of these cells (Hideshima, et al., BLOOD (2003) 101(2):703-705). TNF-alpha-induced IL-6 secretion from BMSCs was also inhibited by the specific p38 MAPK inhibitor.

### SUMMARY OF THE INVENTION

The disclosed invention is directed to methods and compounds useful in treating multiple myeloma (MM) using p38 MAP kinase inhibitors either alone or in combination with other chemotherapeutic compounds. A role for p38 kinase inhibition as a treatment modality for combating multiple myeloma is discussed herein. In a preferred embodiment, small molecule antagonists of p38 MAP kinase are used to treat multiple myeloma.

### BRIEF DESCRIPTION OF THE DRAWINGS

FIG. 1 shows a graphic representation of the effect of a p38 MAPK inhibitor on p38 MAPK phosphorylation in MM cells.

FIG. 2 shows a bar graph showing the effect of a p38 MAPK inhibitor on basal p38 phosphorylation levels in MM cell lines.

FIG. 3 shows the effect of a p38 MAPK inhibitor on p38α MAPK activation and activity in BMSC cells.

FIG. 4 shows the lack of effect of a p38 MAPK inhibitor on BMSC viability.

FIG. 5 shows a line graph illustrating the effect of increasing concentrations of a p38 MAPK inhibitor on MM cell proliferation.

FIG. 6 shows a bar graph illustrating the effects of a p38 MAPK inhibitor on plasma TNFα levels in whole blood.

FIG. 7 shows a bar graph illustrating reduced induction levels of IL-1β in whole blood treated with a p38 MAPK inhibitor.

FIG. 8 shows a bar graph illustrating reduced levels of IL-6 secretion from MM/BMSC cell co-culture.

FIGS. 9A and 9B show bar graphs illustrating the reduction of IL-11 secretion in BMSC (9A) and MM/BMSC (9B) cultures.

FIG. 10 shows a bar graph illustrating that treatment of MM/BMSC co-cultures reduces VEGF secretion.

FIG. 11 shows a line graph illustrating the additive effect of a p38 MAPK inhibitor and the NFκB inhibitor SN-50 in reducing proliferation of MM cells.

### DETAILED DESCRIPTION OF THE PREFERRED EMBODIMENTS

The invention described herein relates to the use of p38 MAP kinase inhibitors, either alone, in combination with other p38 MAP kinase inhibitors, or in combination with other anti-neoplastic agents effective against multiple myeloma (MM). Accordingly, inhibition of p38 MAP kinase activity has a number of direct and indirect effects on MM cells that are therapeutically beneficial for patients suffering from MM.

MAP Kinase Inhibitors, Cytokines and MM

Mitogen-activated protein kinases (MAPKs) are activated by tyrosine and threonine phosphorylation. The disclosed invention has utility in treating MM by modulating MAPKs in to reduce viability of MM cells. One of the key mechanisms by which cell growth and proliferation are regulated involves the mitogenic signal transduction pathway. For example, cell growth is regulated, in part, through the cascade of mitogen-activated protein (MAP) kinase that also includes other transducing molecules such as MAP kinase kinase (MEK) and Raf-1. Constitutive activation of MAP kinases is associated with many cancer cell lines (e.g., pancreas, colon, lung, ovary, and kidney) and primary tumors from various human organs (e.g., kidney, colon, lung), and correlated with the simultaneous expression of MEK and Raf-1 (Hoshino, et al., Oncogene. 18(3):813-22 (1999)). Thus, the level and duration of MAP kinase expression thus appears to control these differential effects. Id.

One family of MAPKs, the p38 MAPK protein kinase family is activated primarily by cellular stresses and not mitogenic stimuli. The activation domain of p38 contains the sequence TGY, which represent the tyrosine and threonine residues required for activation (targeted by MKK3 and MKK6). The physiological role of the different p38 isoforms (which are derived from three genes as well as differential splicing) is still unclear. Among the identified targets for p38 are MAPKAPK-2 and the transcription factors, CHOP/GADD153 (Wang and Ron, Science (1997) 272, 1347-1349), MEF2C (Han et al., Nature (1997), 386, 296-299) and ATF2.

The activation of p38 MAP kinase (phosphorylated form) in MM cells and in bone marrow stromal cells (BMSC) is induced by cytokines and other inflammatory moieties present in the MM bone marrow milieu. Activation of p38 MAP kinase may be induced even in unstimulated MM cells by tumor necrosis factor (TNF). This activation may result in the secretion of cytokines thought to be involved in the pathogenesis of MM. Certain cytokines play a role in promoting a bone marrow microenvironment that is hospitable to the growth, survival, and migration of MM cells. Further, the activated marrow microenvironment in MM supports the unusual drug resistance observed in MM compared to other B cell cancers such as leukemias. Interleukin-6 (IL-6) is thought to be a primary mediator in these effects and is produced by both MM and marrow cells. Additional cytokines thought to play roles in the pathology of MM microenvironment include interleukin-1 (IL-1), interleukin-11 (IL-11), tumor necrosis factor (TNF), insulin-like growth factor-1 (IGF-1), macrophage inflammatory protein-1 (MIP-1), receptor activator of NF-kappa B ligand (RANKL), and transforming growth factor-beta (TGF-β).

Administering MAP kinase inhibitors negatively impacts the bone marrow milieu in which MM cells propagate by altering cytokine expression. For example, p38 inhibitors act to reduce interleukin-6 (IL-6) production from bone marrow stromal cells (BMSCs). Production of IL-6 is thought to be important for maintaining a microenvironment that is favorable for multiple myeloma cell proliferation, that is, MM cell growth and replication. While the impact of p38 inhibitors on cytokine expression, such as IL-6 expression, is a likely mechanism by which to explain the therapeutic impact of p38 inhibitors on MM, it is not the only mechanism available to explain these positive effects. Accordingly, this mechanism is provided solely as a tool for conceptualizing the role that p38 inhibitors can play in treating MM and is not intended to be limiting in any way.

MAP Kinase Inhibitors and MM Cell Drug Resistance

Multiple myeloma is a difficult cancer to treat, in part because MM cells frequently develop resistance to standard chemotherapeutic agents. This is an extremely important point, since patients eventually become resistant to conventional therapy as their disease progresses. The marrow microenvironment in MM is thought to play a role in the unusual resistance to chemotherapy observed for MM cells compared to other B cell cancers.

Multiple myeloma cells cultured from patients retain their initial or treatment-induced resistance profile in many cases. IL-6 expression may be responsible, in part, for dexamethasone resistance of some MM cells. It has already been pointed out that inhibition of the MAP kinase p38 blocks MM cell and BMSC secretion of IL-6. p38 MAP kinase inhibition of IL-6 secretion may thus be an effective means by which to treat MM.

p38 activation may help MM cells adapt in a way that enhances MM cell survival. For example, activated heat shock protein 27 (HSP-27) is thought to play a role in blocking apoptosis in MM cells. Activated p38 phosphorylates and activates HSP-27. By blocking the activation of HSP-27 through p38 inhibition, the postulated anti-apoptotic effect of HSP-27 may be removed. Thus, p38 inhibition may serve to render MM cells sensitive to chemotherapeutic agents, such as apoptosis-promoting agents, to which MM cells might otherwise be resistant.

Inhibitors of p38 MAP Kinase

As used herein, the term “inhibitor” includes, but is not limited to, any suitable molecule, compound, protein or fragment thereof, nucleic acid, formulation or substance that can regulate p38 MAP kinase activity. The inhibitor can affect a single p38 MAP kinase isoform (e.g., p38α, p38β, p38γ or p38δ), more than one isoform, or all isoforms of p38 MAP kinase. In a preferred embodiment, the inhibitor regulates the a isoform of p38 MAP kinase.

In a preferred embodiment of the disclosed invention, it is contemplated that the particular inhibitor can exhibit its regulatory effect upstream or downstream of p38 MAP kinase or on p38 MAP kinase directly. Examples of inhibitor regulated p38 MAP kinase activity include those where the inhibitor can decrease transcription and/or translation of p38 MAP kinase, can decrease or inhibit post-translational modification and/or cellular trafficking of p38 MAP kinase, or can shorten the half-life of p38 MAP kinase. The inhibitor can also reversibly or irreversibly bind p38 MAP kinase, inactivate its enzymatic activity, or otherwise interfere with its interaction with downstream substrates.

If acting on p38 MAP kinase directly, in one embodiment the inhibitor should exhibit an IC₅₀ value of about 5 μM or less, preferably about 500 mM or less, more preferably about 100 nM or less. In a related embodiment, the inhibitor should exhibit an IC₅₀ value relative to the p38α MAP kinase isoform that is about ten fold less than that observed when the same inhibitor is tested against other p38 MAP kinase isoforms in a comparable assay.

Those skilled in the art can determine whether or not a compound is useful in the disclosed invention by evaluating its p38 MAP kinase activity relative to its IC₅₀ value for p38 kinase. This evaluation can be accomplished through conventional in vitro assays. In vitro assays include assays that assess inhibition of kinase or ATPase activity of activated p38 MAP kinase. In vitro assays can also assess the ability of the inhibitor to bind to a p38 MAP kinase or to reduce or block an identified downstream effect of the activated p38 MAP kinase, e.g., cytokine secretion. IC₅₀ values are calculated using the concentration of inhibitor that causes a 50% decrease as compared to a control.

A binding assay is a fairly inexpensive and simple in vitro assay to run. As previously mentioned, binding of a molecule to p38 MAP kinase, in and of itself, can be inhibitory, due to steric, allosteric or charge-charge interactions. A binding assay can be performed in solution or on a solid phase using p38 MAP kinase or a fragment thereof as a target. By using this as an initial screen, one can evaluate libraries of compounds for potential p38 MAP kinase regulatory activity.

The target in a binding assay can be either free in solution, fixed to a support, or expressed in or on the surface of a cell. A label (e.g., radioactive, fluorescent, quenching, etc.) can be placed on the target, compound, or both to determine the presence or absence of binding. This approach can also be used to conduct a competitive binding assay to assess the inhibition of binding of a target to a natural or artificial substrate or binding partner. In any case, one can measure, either directly or indirectly, the amount of free label versus bound label to determine binding. There are many known variations and adaptations of this approach to minimize interference with binding activity and optimize signal.

For purposes of in vitro cellular assays, the compounds that represent potential inhibitors of p38 MAP kinase function can be administered to a cell in any number of ways. Preferably, the compound or composition can be added to the medium in which the cell is growing, such as tissue culture medium for cells grown in culture. The compound is provided in standard serial dilutions or in an amount determined by analogy to known modulators. Alternatively, the potential inhibitor can be encoded by a nucleic acid that is introduced into the cell wherein the cell produces the potential inhibitor itself.

Alternative assays involving in vitro analysis of potential inhibitors include those where cells (e.g., HeLa) transfected with DNA coding for relevant kinases can be activated with substances such as sorbitol, IL-1, TNF, or PMA. After immunoprecipitation of cell lysates, equal aliquots of immune complexes of the kinases are pre-incubated for an adequate time with a specific concentration of the potential inhibitor followed by addition of kinase substrate buffer mix containing labeled ATP and GST-ATF2 or MBP. After incubation, kinase reactions are terminated by the addition of SDS loading buffer. Phosphorylated substrate is resolved through SDS-PAGE and visualized and quantitated in a phosphorimager. The p38 MAP kinase regulation, in terms of phosphorylation and IC₅₀ values, can be determined by quantitation. See e.g., Kumar, S. et al., Biochem. Biophys. Res. Commun. 235:533-538 (1997). Similar techniques can be used to evaluate the effects of potential inhibitors on other MAP kinases.

Other in vitro assays can assess the production of TNF-α as a correlation to p38 MAP kinase activity. One such example is a Human Whole Blood Assay. In this assay, venous blood is collected from, e.g., healthy male volunteers into a heparinized syringe and is used within 2 hours of collection. Test compounds are dissolved in 100% DMSO and 1 μl aliquots of drug concentrations ranging from 0 to 1 mM are dispensed into quadruplicate wells of a 24-well microtiter plate (Nunclon Delta SI, Applied Scientific Co., San Francisco, Calif.). Whole blood is added at a volume of 1 ml/well and the mixture is incubated for 15 minutes with constant shaking (Titer Plate Shaker, Lab-Line Instruments, Inc., Melrose Park, Ill.) at a humidified atmosphere of 5% CO₂ at 37° C. Whole blood is cultured either undiluted or at a final dilution of 1:10 with RPMI 1640 (Gibco 31800+NaHCO₃, Life Technologies, Rockville, Md. and Scios, Inc., Sunnyvale, Calif.). At the end of the incubation period, 10 μl of LPS (E. coli 01111:B4, Sigma Chemical Co., St. Louis, Mo.) is added to each well to a final concentration of 1 or 0.1 μg/ml for undiluted or 1:10 diluted whole blood, respectively. The incubation is continued for an additional 2 hours. The reaction is stopped by placing the microtiter plates in an ice bath, and plasma or cell-free supernates are collected by centrifugation at 3000 rpm for 10 minutes at 4° C. The plasma samples are stored at −80° C. until assayed for TNF-α levels by ELISA, following the directions supplied by Quantikine Human TNF-α assay kit (R&D Systems, Minneapolis, Minn.). IC₅₀ values are calculated using the concentration of inhibitor that causes a 50% decrease as compared to a control.

A similar assay is an Enriched Mononuclear Cell Assay. The enriched mononuclear cell assay begins with cryopreserved Human Peripheral Blood Mononuclear Cells (HPBMCs) (Clonetics Corp.) that are rinsed and resuspended in a warm mixture of cell growth media. The resuspended cells are then counted and seeded at 1×10⁶ cells/well in a 24-well microtitre plate. The plates are then placed in an incubator for an hour to allow the cells to settle in each well. After the cells have settled, the media is aspirated and new media containing 100 ng/ml of the cytokine stimulatory factor Lipopolysaccharide (LPS) and a test chemical compound is added to each well of the microtiter plate. Thus, each well contains HPBMCs, LPS and a test chemical compound. The cells are then incubated for 2 hours, and the amount of the cytokine Tumor Necrosis Factor Alpha (TNF-α) is measured using an Enzyme Linked Immunoassay (ELISA). One such ELISA for detecting the levels of TNF-α is commercially available from R&D Systems. The amount of TNF-α production by the HPBMCs in each well is then compared to a control well to determine whether the chemical compound acts as an inhibitor of cytokine production.

While IC₅₀ values are an initial indicia for identifying compounds that are useful for the invention, it is contemplated that one skilled in the art would further consider additional and conventional pharmaceutical considerations including but not limited to bioavailability, pK values, routes of delivery, solubility, and the like.

Exemplary Inhibitors

Preferred examples of the compounds of the invention are of the formula:

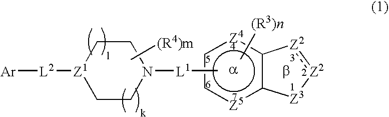

and the pharmaceutically acceptable salts thereof, or a pharmaceutical composition thereof, wherein

[symbol] represents a single or double bond;

one Z² is CA or CR⁸A and the other is CR¹, CR¹₂, NR⁶ or N wherein each R¹, R⁶ and R⁸ is independently hydrogen or noninterfering substituent;

A is —Wᵢ—COXⱼY wherein Y is COR² or an isostere thereof and R² is hydrogen or a noninterfering substituent, each of W and X is a spacer of 2-6 Å, and each of i and j is independently 0 or 1;

Z³ is NR⁷ or O;

each of Z⁴ and Z⁵ is independently N or CR¹ wherein R¹ is as defined above and wherein at least one of Z⁴ and Z⁵ is N;

each R³ is independently a noninterfering substituent;

n is 0-3;

each of L¹ and L² is a linker;
each R⁴ is independently a noninterfering substituent;

m is 0-4;

Z¹ is CR⁵ or N wherein R⁵ is hydrogen or a noninterfering substituent;

each of l and k is an integer from 0-2 wherein the sum of l and k is 0-3;

Ar is an aryl group substituted with 0-5 noninterfering substituents, wherein two noninterfering substituents can form a fused ring.

Preferred embodiments of compounds useful in the invention are derivatives of indole-type compounds containing a mandatory substituent, A, at a position corresponding to the 2- or 3-position of indole. In general, an indole-type nucleus is preferred, although alternatives within the scope of the invention are also illustrated below. Additionally, PCT publication WO00/71535, published 7 Dec. 2000, discloses indole derived compounds that are specific inhibitors of p38 kinase. The disclosure of this document is incorporated herein by reference.

U.S. Provisional Patent Application No. 60/417,599 filed 9 Oct. 2002 and U.S. patent application Ser. No. 10/683,656, filed Oct. 9, 2003, disclose azaindole derivatives that are useful in treating conditions that are characterized by enhanced p38 activity and are therefore useful for purposes of this invention. The disclosure of these documents is incorporated herein by reference.

As used herein, a “noninterfering substituent” is a substituent which either leaves the ability of the compound of formula (1) to inhibit p38-α activity qualitatively intact or enhances the activity of the inhibitor. Thus, the substituent may alter the degree of inhibition of p38 However, as long as the compound of formula (1) retains the ability to inhibit p38 activity, the substituent will be classified as “noninterfering.” As mentioned above, a number of assays for determining the ability of any compound to inhibit p38 activity are available in the art. A whole blood assay for this evaluation is illustrated below: the gene for p38 has been cloned and the protein can be prepared recombinantly and its activity assessed, including an assessment of the ability of an arbitrarily chosen compound to interfere with this activity. The essential features of the molecule are tightly defined. The positions which are occupied by “noninterfering substituents” can be substituted by conventional organic moieties as is understood in the art. It is irrelevant to the present invention to test the outer limits of such substitutions.

Regarding the compounds of formula (1), L¹ and L² are described herein as linkers. Typical linkers include alkylene, i.e. (CH₂)ₙ—R; alkenylene—i.e., an alkylene moiety which contains a double bond, including a double bond at one terminus. Other suitable linkers include, for example, substituted alkylenes or alkenylenes, carbonyl moieties, and the like.

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when so stated however, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or contain heteroatoms within the “backbone” of the hydrocarbyl residue.

As used herein, “inorganic residue” refers to a residue that does not contain carbon. Examples include, but are not limited to, halo, hydroxy, NO₂ or NH₂.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-20, S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aromatic” moiety refers to a monocyclic or fused bicyclic moiety such as phenyl or naphthyl; “heteroaromatic” also refers to monocyclic or fused bicyclic ring systems containing one or more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroalkyl” refer to aromatic and heteroaromatic systems which are coupled to another residue through a carbon chain, including substituted or unsubstituted, saturated or unsaturated, carbon chains, typically of 1-6C. These carbon chains may also include a carbonyl group, thus making them able to provide substituents as an acyl moiety.

When the compounds of Formula 1 contain one or more chiral centers, the invention includes optically pure forms as well as mixtures of stereoisomers or enantiomers.

With respect to the portion of the compound of formula (1) between the atom of Ar bound to L² and ring a, L¹ and L² are linkers which space the substituent Ar from ring a at a distance of 4.5-24 Å, preferably 6-20 Å, more preferably 7.5-10 Å. In a preferred embodiment, the distance of substituent Ar from ring is less than 24 Å. The distance is measured from the center of the a ring to the atom of Ar to which the linker L² is attached. Typical, but nonlimiting, embodiments of L¹ and L² are CO and isosteres thereof, or optionally substituted isosteres, or longer chain forms. L², in particular, may be alkylene or alkenylene optionally substituted with noninterfering substituents or L¹ or L² may be or may include a heteroatom such as N, S or O. Such substituents include, but are limited to, a moiety selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two substituents on L² can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety.

Isosteres of CO and CH₂, include SO, SO₂, or CHOH. CO and CH₂ are preferred.

Thus, L² is substituted with 0-2 substituents. Where appropriate, two optional substituents on L can be joined to form a non-aromatic saturated or unsaturated hydrocarbyl ring that includes 0-3 heteroatoms such as O, S and/or N and which contains 3 to 8 members. Two optional substituents on L² can be joined to form a carbonyl moiety which can be subsequently converted to an oxime, an oximeether, an oximeester, or a ketal.

Ar is aryl, heteroaryl, including 6-5 fused heteroaryl, cycloaliphatic or cycloheteroaliphatic that can be optionally substituted. Ar is preferably optionally substituted phenyl.

Each substituent on Ar is independently a hydrocarbyl residue (1-20C) containing 0-5 heteroatoms selected from O, S and N, or is an inorganic residue. Preferred substituents include those selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof, and wherein two of said optional substituents on adjacent positions can be joined to form a fused, optionally substituted aromatic or nonaromatic, saturated or unsaturated ring which contains 3-8 members. More preferred substituents include halo, alkyl (1-4C) and more preferably, fluoro, chloro and methyl. These substituents may occupy all available positions of the aryl ring of Ar, preferably 1-2 positions, most preferably one position. These substituents may be optionally substituted with substituents similar to those listed. Of course some substituents, such as halo, are not further substituted, as known to one skilled in the art.

Two substituents on Ar can be joined to form a fused, optionally substituted aromatic or nonaromatic, saturated or unsaturated ring which contains 3-8 members.

Regarding formula (1), between L¹ and L² is a piperidine-type moiety of the following formula:

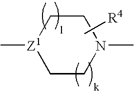

Z¹ is CR⁵ or N wherein R⁵ is H or a noninterfering substituent. Each of l and k is an integer from 0-2 wherein the sum of l and k is 0-3. The noninterfering substituents R⁵ include, without limitation, halo, alkyl, alkoxy, aryl, arylalkyl, aryloxy, heteroaryl, acyl, carboxy, or hydroxy. Preferably, R⁵ is H, alkyl, OR, NR₂, SR or halo, where R is H or alkyl. Additionally, R⁵ can be joined with an R⁴ substituent to form an optionally substituted non-aromatic saturated or unsaturated hydrocarbyl ring which contains 3-8 members and 0-3 heteroatoms such as O, N and/or S. Preferred embodiments include compounds wherein Z¹ is CH or N, and those wherein both l and k are 1.

R⁴ represents a noninterfering substituent such as a hydrocarbyl residue (1-20C) containing 0-5 heteroatoms selected from O, S and N. Preferably R⁴ is alkyl, alkoxy, aryl, arylalkyl, aryloxy, heteroalkyl, heteroaryl, heteroarylalkyl, RCO, ═O, acyl, halo, CN, OR, NRCOR, NR, wherein R is H, alkyl (preferably 1-4C), aryl, or hetero forms thereof. Each appropriate substituent is itself unsubstituted or substituted with 1-3 substituents. The substituents are preferably independently selected from a group that includes alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof. R⁴ may occur m times on the ring; m is an integer of 0-4. Preferred embodiments of R⁴ comprise alkyl (1-4C) especially two alkyl substituents and carbonyl. Most preferably R⁴ comprises two methyl groups at positions 2 and 5 or 3 and 6 of a piperidinyl or piperazinyl ring or ═O preferably at the 5-position of the ring. The substituted forms may be chiral and an isolated enantiomer may be preferred.

R³ also represents a noninterfering substituent. Such substituents include hydrocarbyl residues (1-6C) containing 0-2 heteroatoms selected from O, S and/or N and inorganic residues. n is an integer of 0-3, preferably 0 or 1. Preferably, the substituents represented by R³ are independently halo, alkyl, heteroalkyl, OCOR, OR, NRCOR, SR, or NR₂, wherein R is H, alkyl, aryl, or heteroforms thereof. More preferably R³ substituents are selected from alkyl, alkoxy or halo, and most preferably methoxy, methyl, and chloro. Most preferably, n is 0 and the a ring is unsubstituted, except for L¹ or n is 1 and R³ is halo or methoxy.

In the ring labeled β, Z³ may be NR⁷ or O— i.e., the compounds may be related to indole or benzofuran. If C³ is NR⁷, preferred embodiments of R⁷ include H or optionally substituted alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof. More preferably, R⁷ is hydrogen or is alkyl (1-4C), preferably methyl or is acyl (1-4C), or is COOR wherein R is H, alkyl, alkenyl of aryl or hetero forms thereof. R⁷ is also preferably a substituted alkyl wherein the preferred substituents are form ether linkages or contain sulfinic or sulfonic acid moieties. Other preferred substituents include sulfhydryl substituted alkyl substituents. Still other preferred substituents include CONR₂ wherein R is defined as above.

It is preferred that the indicated dotted line represents a double bond; however, compounds which contain a saturated β ring are also included within the scope of the invention.

Preferably, the mandatory substituent CA or CR⁸A is in the 3-position; regardless of which position this substituent occupies, the other position is CR¹, CR¹₂, NR⁶ or N. CR¹ is preferred. Preferred embodiments of R¹ include hydrogen, alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOR, SO₃R, CONR₂, SO₂NR₂, NRSO₂NR₂, CN, CF₃, R₃Si, and NO₂, wherein each R is independently H, alkyl, alkenyl or aryl or heteroforms thereof and two of R¹ can be joined to form a fused, optionally substituted aromatic or nonaromatic, saturated or unsaturated ring which contains 3-8 members. Most preferably, R¹ is H, alkyl, such as methyl, most preferably, the ring labeled a contains a double bond and CR¹ is CH or C-alkyl. Other preferable forms of R¹ include H, alkyl, acyl, aryl, arylalkyl, heteroalkyl, heteroaryl, halo, OR, NR₂, SR, NRCOR, alkyl-OOR, RCO, COOR, and CN, wherein each R is independently H, alkyl, or aryl or heteroforms thereof.

While the position not occupied by CA is preferred to include CR¹, the position can also be N or NR⁶. While NR⁶ is less preferred (as in that case the ring labeled β would be saturated), if NR⁶ is present, preferred embodiments of R⁶ include H, or alkyl, alkenyl, alkynyl, aryl, arylalkyl, acyl, aroyl, heteroaryl, heteroalkyl, heteroalkenyl, heteroalkynyl, heteroalkylaryl, or is SOR, SO₂R, RCO, COOR, alkyl-COR, SO₃R, CONR₂, SO₂NR₂, CN, CF₃, or R₃Si wherein each R is independently H, alkyl, alkenyl or aryl or heteroforms thereof.

Preferably, CR⁸A or CA occupy position 3- and preferably Z² in that position is CA. However, if the β ring is saturated and R⁸ is present, preferred embodiments for R⁸ include H, halo, alkyl, alkenyl and the like. Preferably R⁸ is a relatively small substituent corresponding, for example, to H or lower alkyl 1-4C.

A is —Wᵢ—COXⱼY wherein Y is COR² or an isostere thereof and R² is a noninterfering substituent. Each of W and X is a spacer and may be, for example, optionally substituted alkyl, alkenyl, or alkynyl, each of i and j is 0 or 1. Preferably, W and X are unsubstituted. Preferably, j is 0 so that the two carbonyl groups are adjacent to each other. Preferably, also, i is 0 so that the proximal CO is adjacent the ring. However, compounds wherein the proximal CO is spaced from the ring can readily be prepared by selective reduction of an initially glyoxal substituted β ring. In the most preferred embodiments of the invention, the α/β ring system is an indole containing CA in position 3- and wherein A is COCR².

The noninterfering substituent represented by R², when R² is other than H, is a hydrocarbyl residue (1-20C) containing 0-5 heteroatoms selected from O, S and/or N or is an inorganic residue. Preferred are embodiments wherein R² is H, or is straight or branched chain alkyl, alkenyl, alkynyl, aryl, arylalkyl, heteroalkyl, heteroaryl, or heteroarylalkyl, each optionally substituted with halo, alkyl, heteroalkyl, SR, OR, NR₂, OCOR, NRCOR, NRCONR₂, NRSO₂R, NRSO₂NR₂, OCONR₂, CN, COOR, CONR₂, COR, or R₃Si wherein each R is independently H, alkyl, alkenyl or aryl or the heteroatom-containing forms thereof, or wherein R² is OR, NR₂, SR, NRCONR₂, OCONR₂, or NRSO₂NR₂, wherein each R is independently H, alkyl, alkenyl or aryl or the heteroatom-containing forms thereof, and wherein two R attached to the same atom may form a 3-8 member ring and wherein said ring may further be substituted by alkyl, alkenyl, alkynyl, aryl, arylalkyl, heteroalkyl, heteroaryl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroatom-containing forms thereof wherein two R attached to the same atom may form a 3-8 member ring, optionally substituted as above defined.

Other preferred embodiments of R² are H, heteroarylalkyl, —NR₂, heteroaryl, —COOR, —NHRNR₂, heteroaryl-COOR, heteroaryloxy, —OR, heteroaryl-NR₂, —NROR and alkyl. Most preferably R² is isopropyl piperazinyl, methyl piperazinyl, dimethylamine, piperazinyl, isobutyl carboxylate, oxycarbonylethyl, morpholinyl, aminoethyldimethylamine, isobutyl carboxylate piperazinyl, oxypiperazinyl, ethylcarboxylate piperazinyl, methoxy, ethoxy, hydroxy, methyl, amine, aminoethyl pyrrolidinyl, aminopropanediol, piperidinyl, pyrrolidinyl-piperidinyl, or methyl piperidinyl.

Isosteres of COR² as represented by Y are defined as follows.

The isosteres have varying lipophilicity and may contribute to enhanced metabolic stability. Thus, Y, as shown, may be replaced by the isosteres in Table 1.

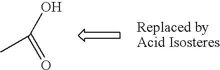

TABLE 1
Acid Isosteres

| Names | | |
|---|---|---|
| of Groups | Chemical Structures | Substitution Groups (SG) |
| tetrazole | 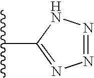 | n/a |
| 1,2,3-triazole | 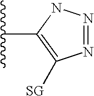 | H; SCH₃; COCH₃; Br; SOCH₃; SO₂CH₃; NO₂; CF₃; CN; COOMe |
| 1,2,4-triazole | 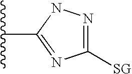 | H; SCH₃; COCH₃; Br; SOCH₃; SO₂CH₃; NO₂ |
| imidazole | 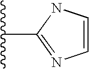 | H; SCH₃; COCH₃; Br; SOCH₃; SO₂CH₃; NO₂ |

Thus, isosteres include tetrazole, 1,2,3-triazole, 1,2,4-triazole and imidazole.

The compounds of formula (1) may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (1), the compound may also be supplied as a salt with a pharmaceutically acceptable cation.

Compounds useful in the practice of the disclosed invention include, but are not limited to, the compounds shown in Table 2, below.

TABLE 2
Exemplary p38 Inhibitors

| Cpd. # | Mol. Structure |
|---|---|
| 1 | 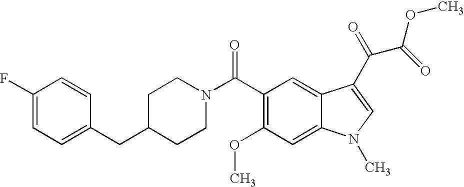 |
| 2 | 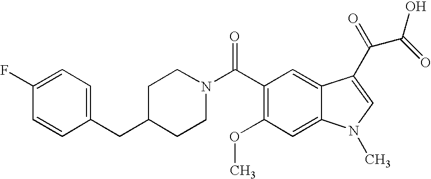 |
| 3 | 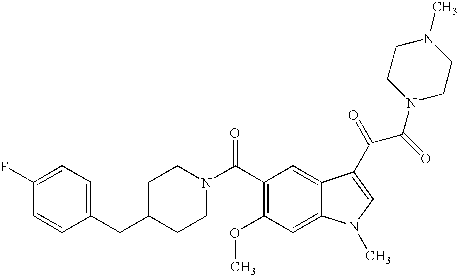 |
| 4 | 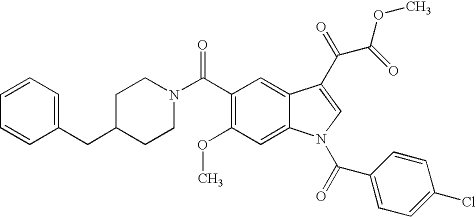 |
| 5 | 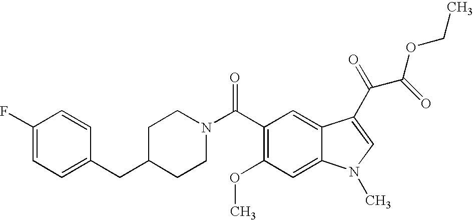 |
| 6 | 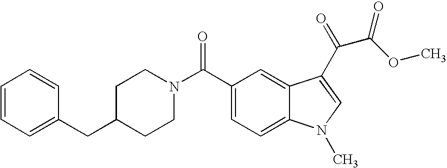 |
| 7 | 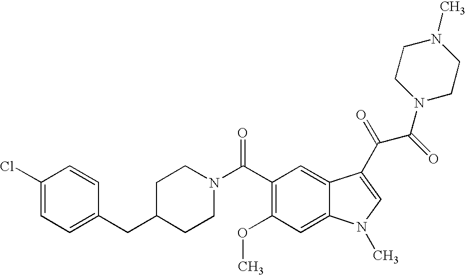 |
| 8 | 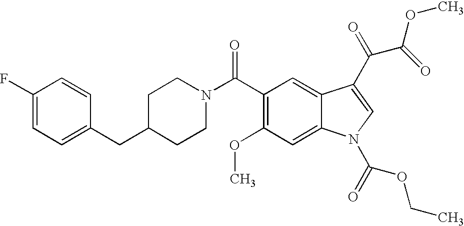 |
| 9 | 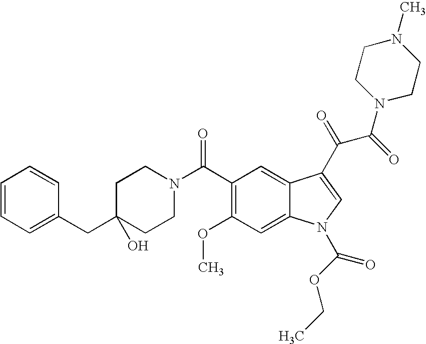 |
| 10 | 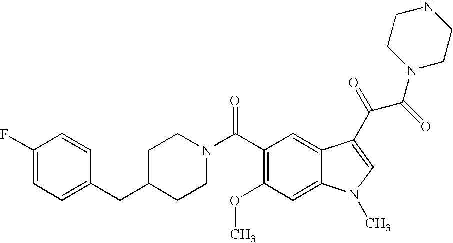 |
| 11 | 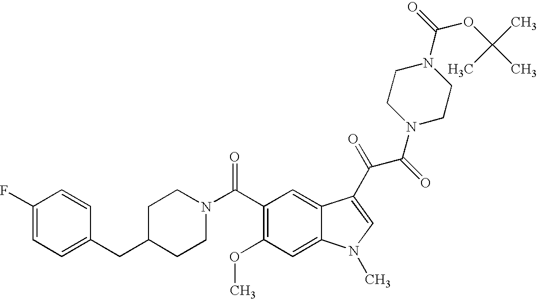 |
| 12 | 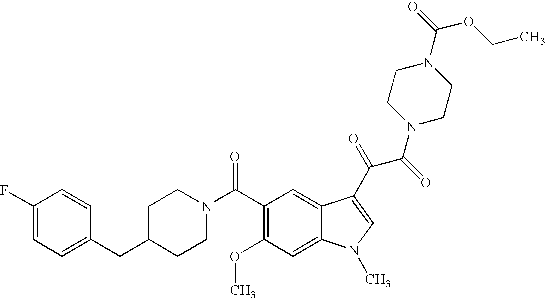 |
| 13 | 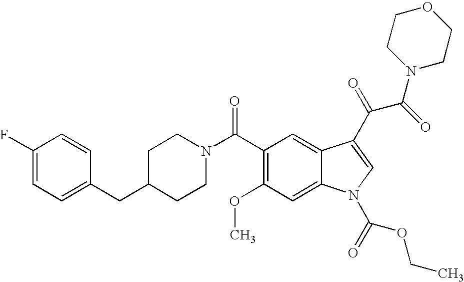 |
| 14 | 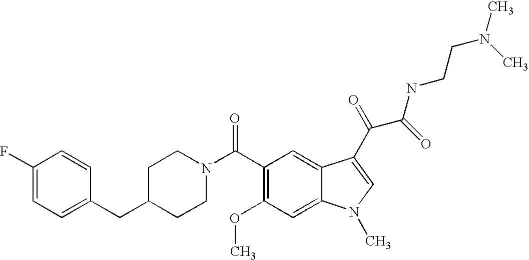 |
| 15 | 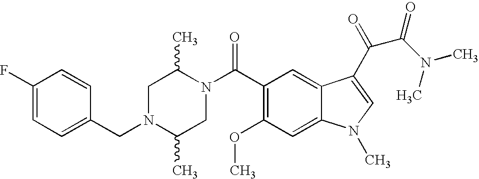 |
| 16 | 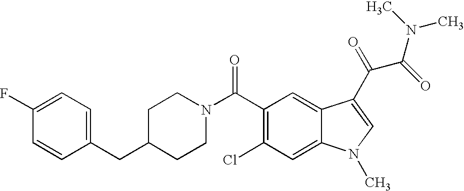 |
| 17 | 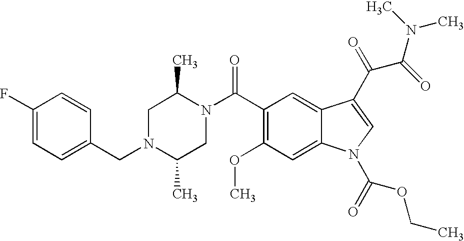 |
| 18 | 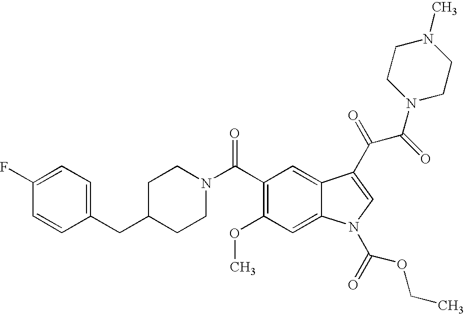 |
| 19 | 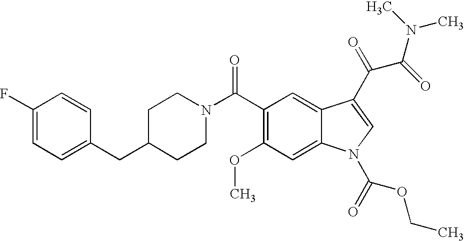 |
| 20 | 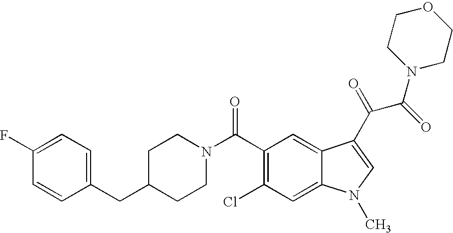 |
| 21 | 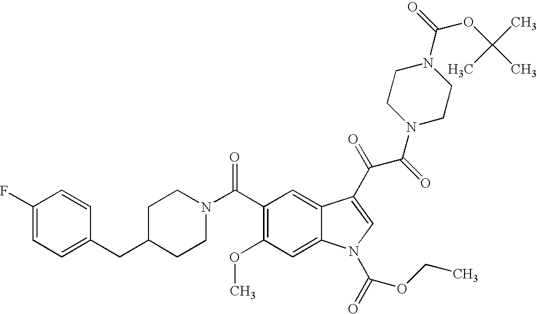 |
| 22 | 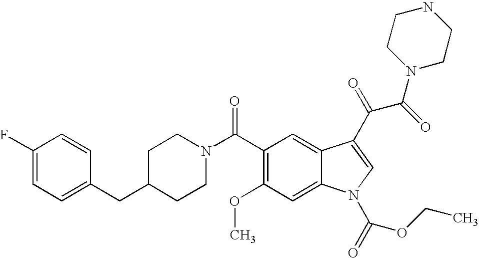 |
| 23 | 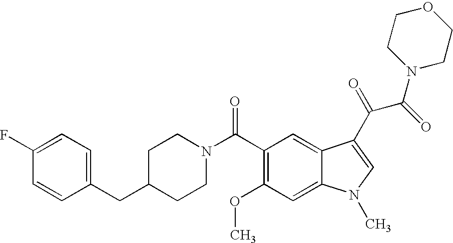 |
| 24 | 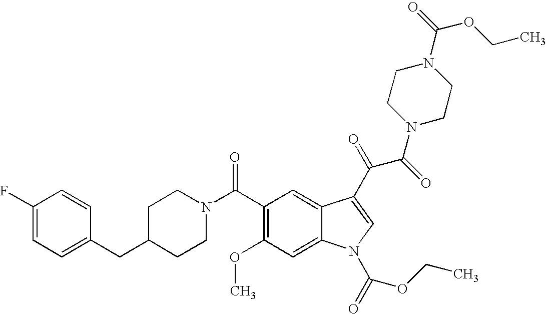 |
| 25 | 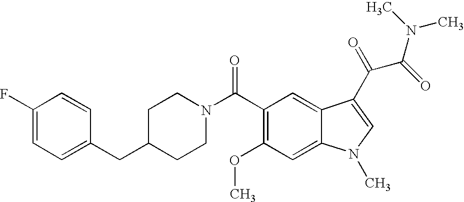 |
| 26 | 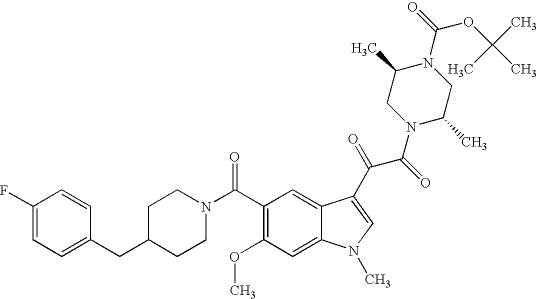 |
| 27 | 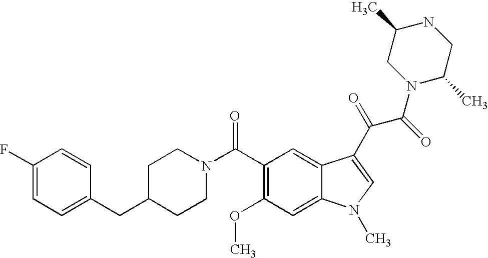 |
| 28 | 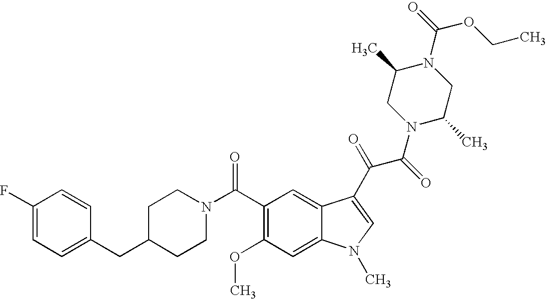 |
| 29 | 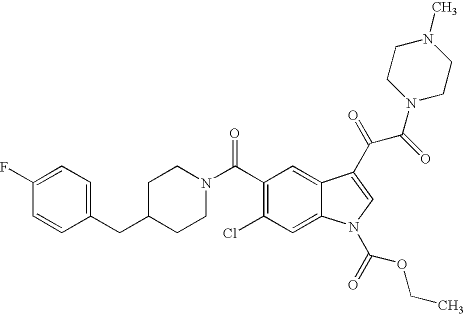 |
| 30 | 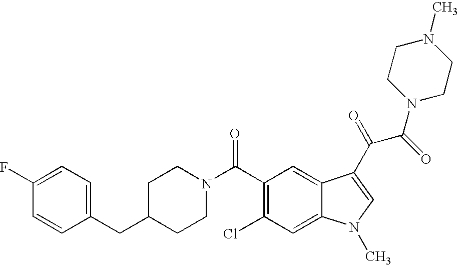 |
| 31 | 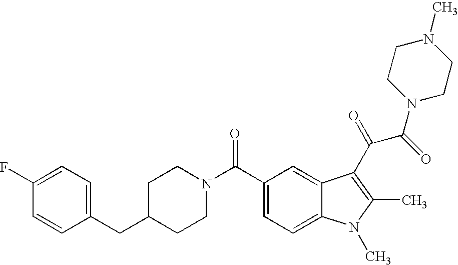 |
| 32 | 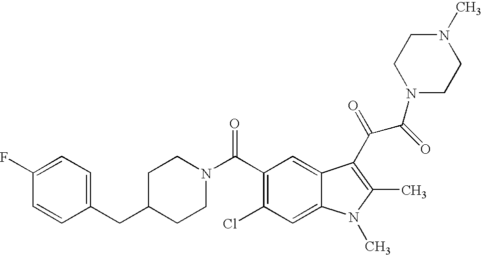 |
| 33 | 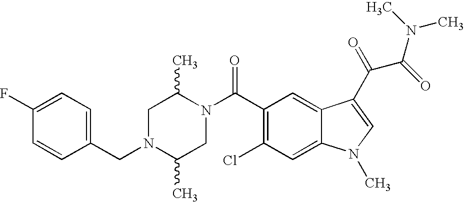 |
| 34 | 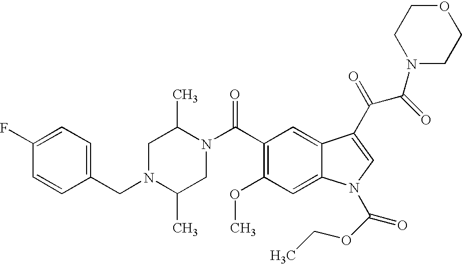 |
| 35 | 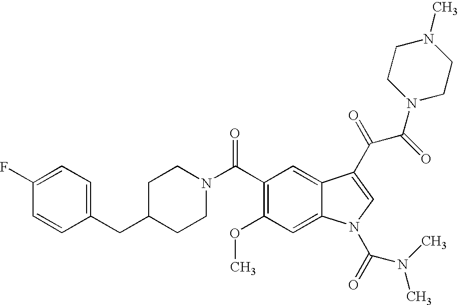 |
| 36 | 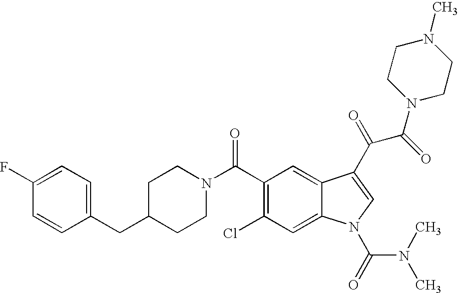 |
| 37 | 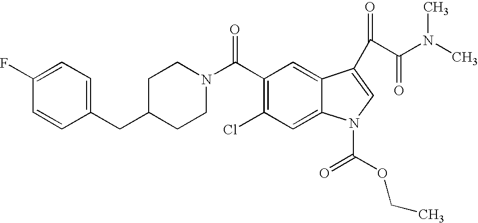 |
| 38 | 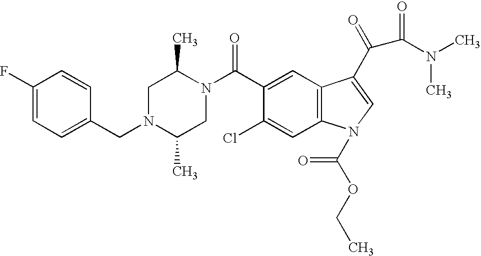 |
| 39 | 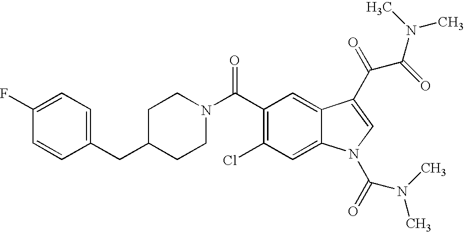 |
| 40 | 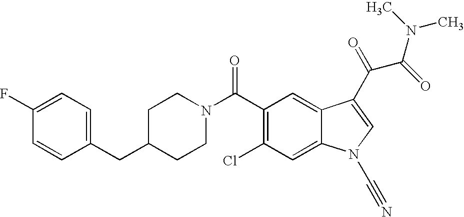 |
| 41 | 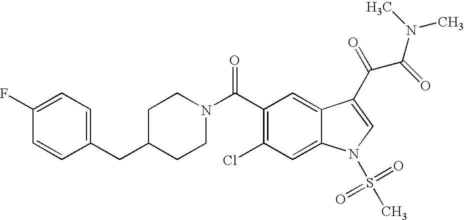 |
| 42 | 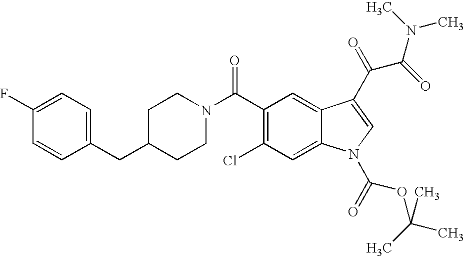 |
| 43 | 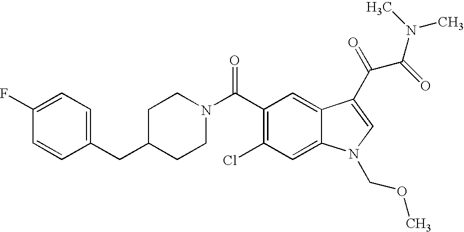 |
| 44 | 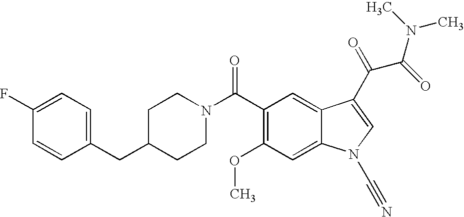 |
| 45 | 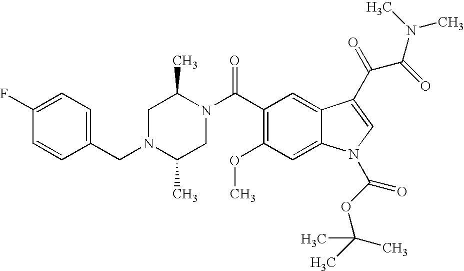 |
| 46 | 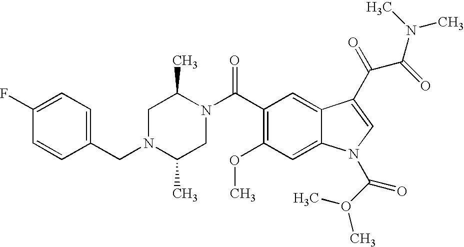 |
| 47 | 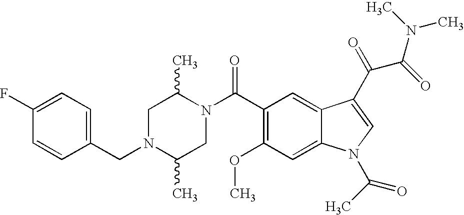 |
| 48 | 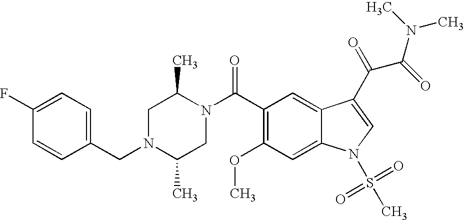 |
| 49 | 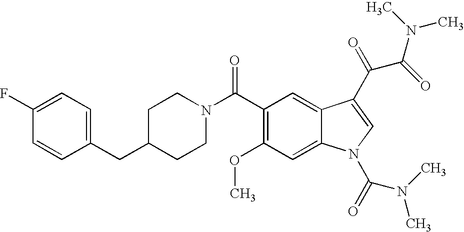 |
| 50 | 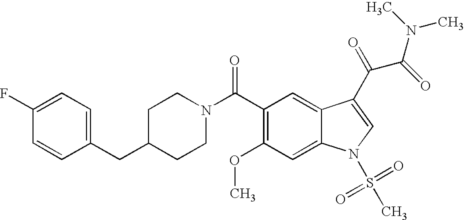 |
| 51 | 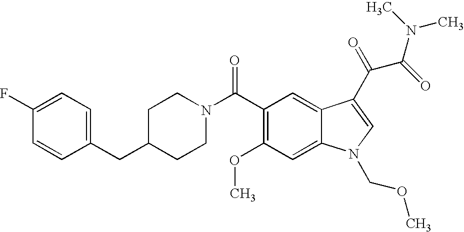 |
| 52 | 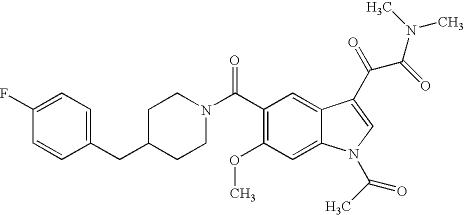 |
| 53 | 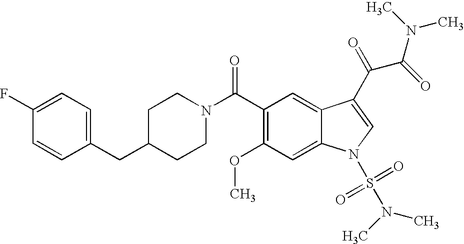 |
| 54 | 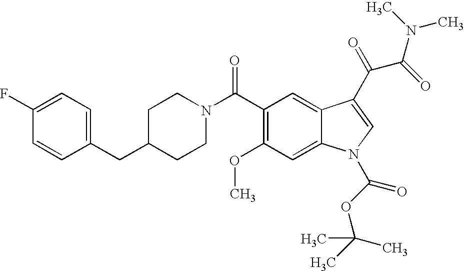 |
| 55 | 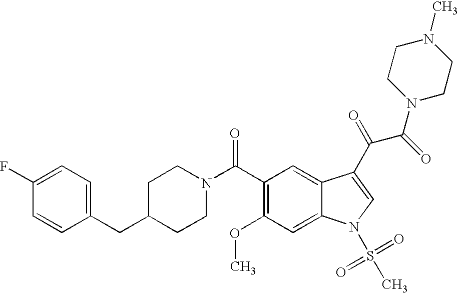 |
| 56 | 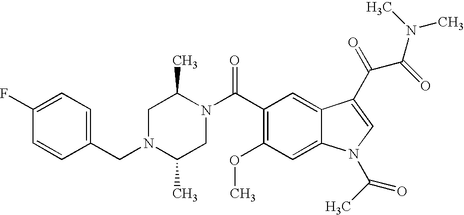 |
| 57 | 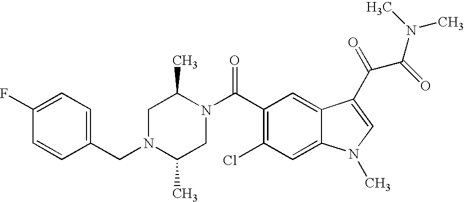 |
| 58 | 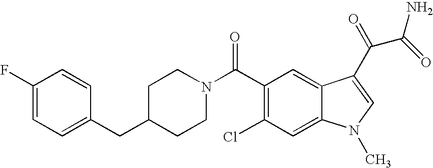 |
| 59 | 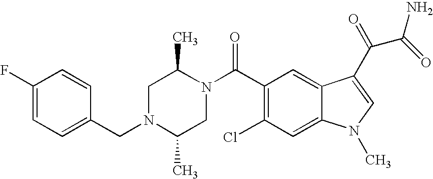 |
| 60 | 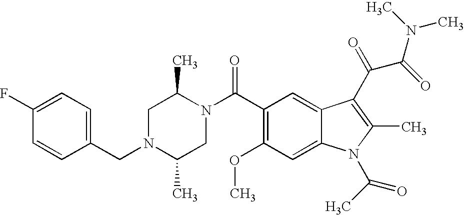 |
| 61 | 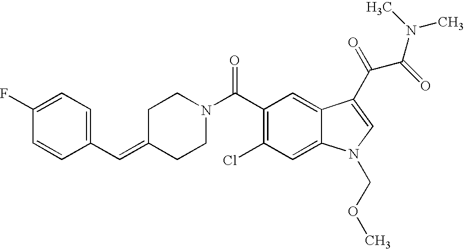 |
| 62 | 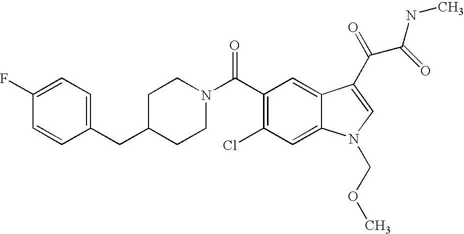 |
| 63 | 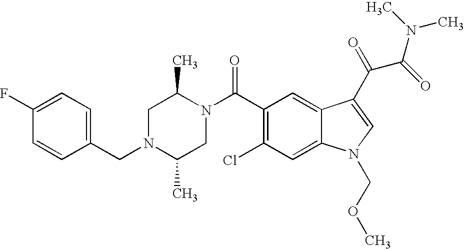 |
| 64 | 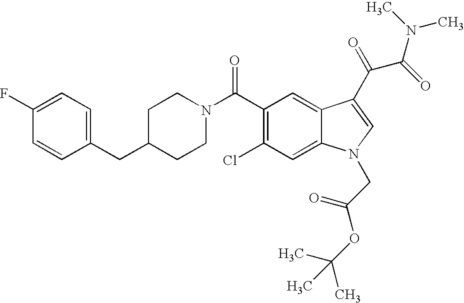 |
| 65 | 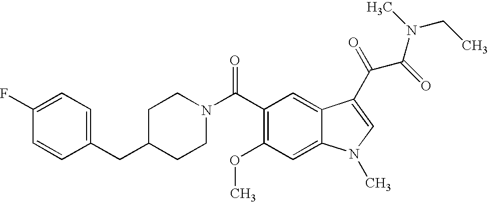 |
| 66 | 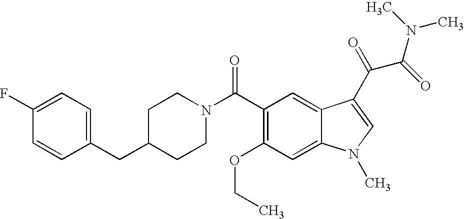 |
| 67 | 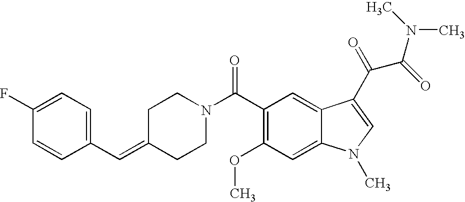 |
| 68 | 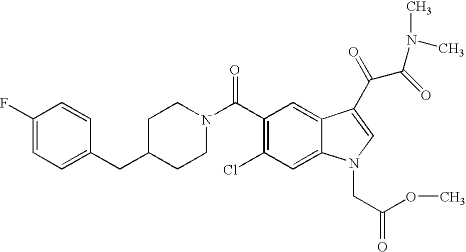 |
| 69 | 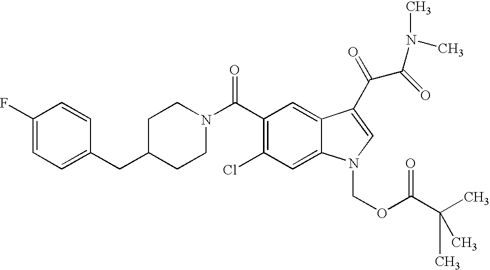 |
| 70 | 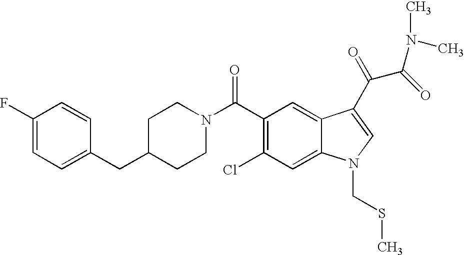 |
| 71 | 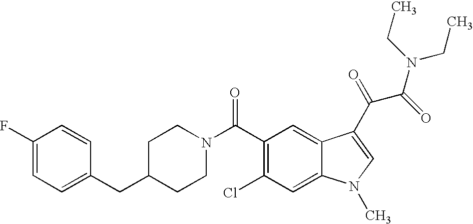 |
| 72 | 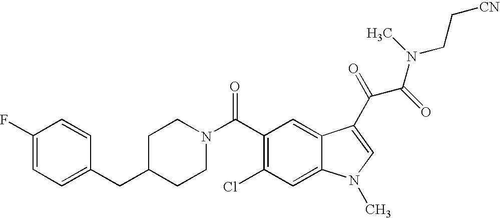 |
| 73 | 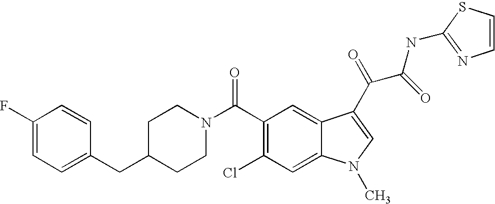 |
| 74 | 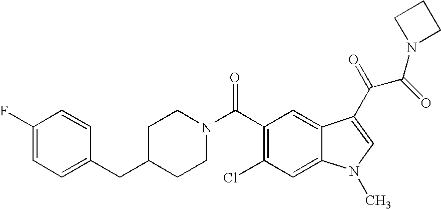 |
| 75 | 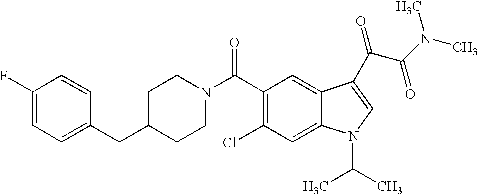 |
| 76 | 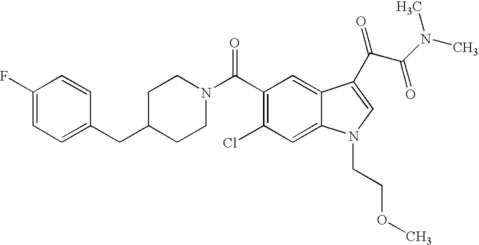 |
| 77 | 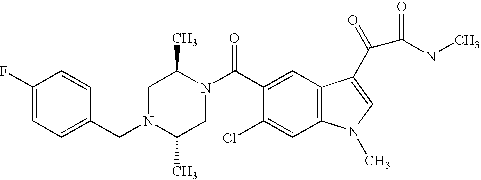 |
| 78 | 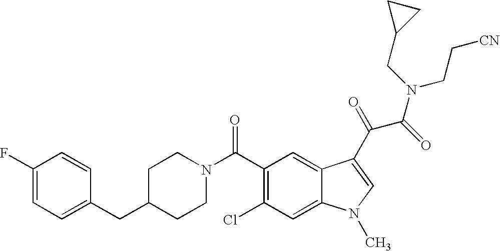 |
| 79 | 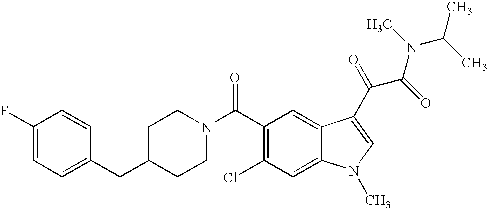 |
| 80 | 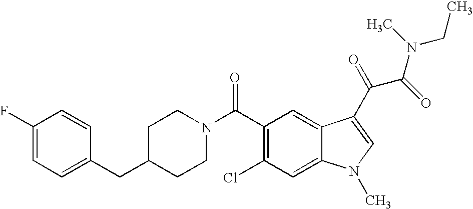 |
| 81 | 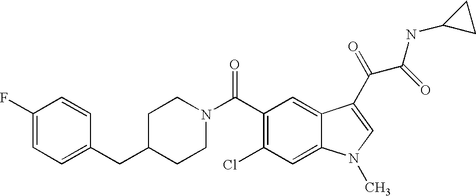 |
| 82 | 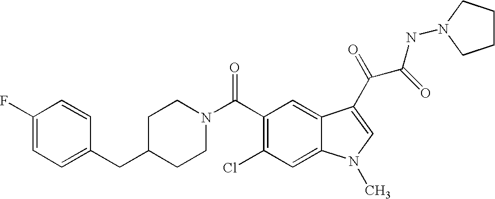 |
| 83 | 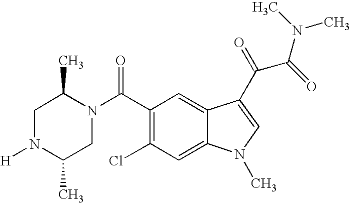 |
| 84 | 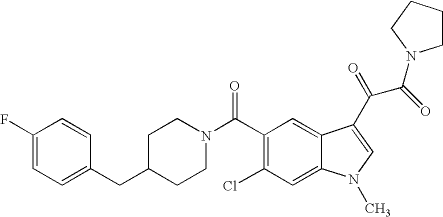 |
| 85 | 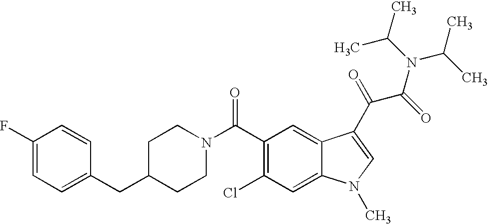 |
| 86 | 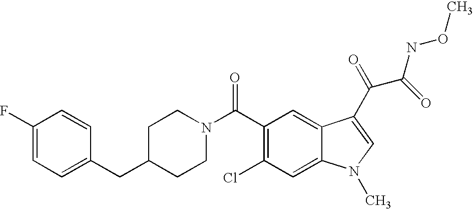 |
| 87 | 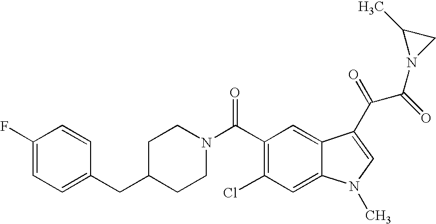 |
| 88 | 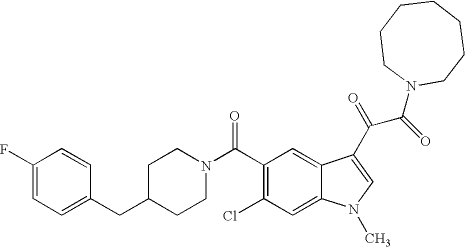 |
| 89 | 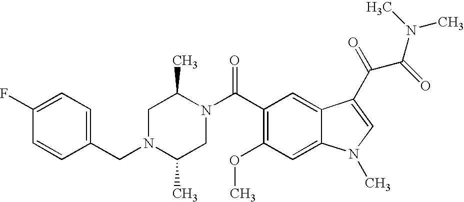 |
| 90 | 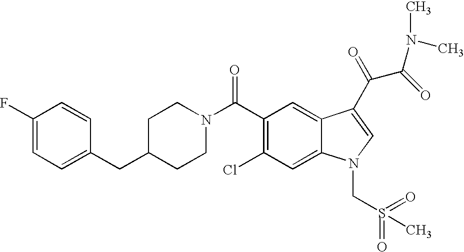 |
| 91 | 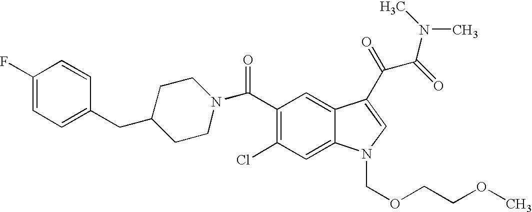 |
| 92 | 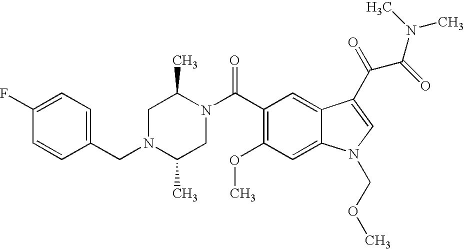 |
| 93 | 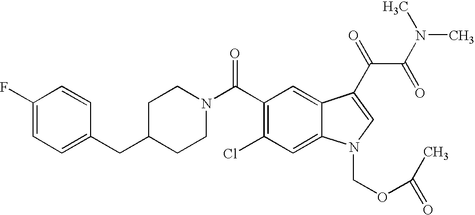 |
| 94 | 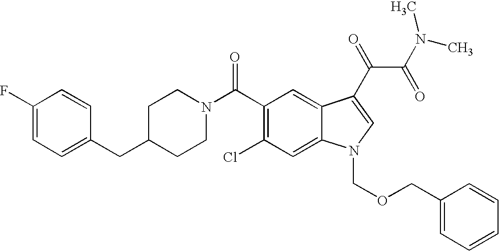 |
| 95 | 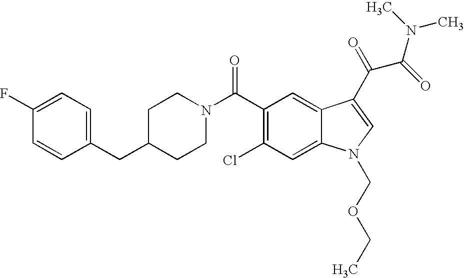 |
| 96 | 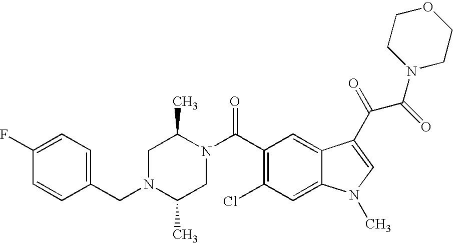 |
| 97 | 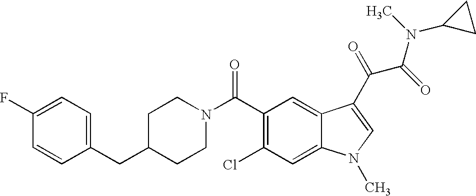 |
| 98 | 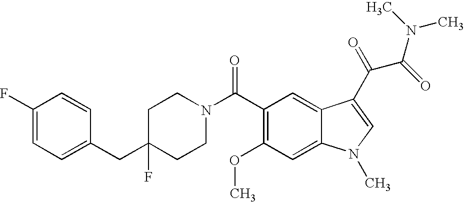 |
| 99 | 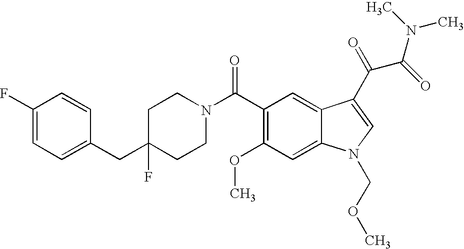 |
| 100 | 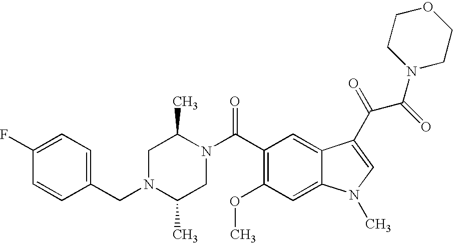 |
| 101 | 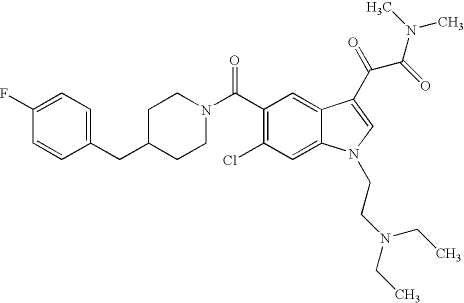 |
| 102 | 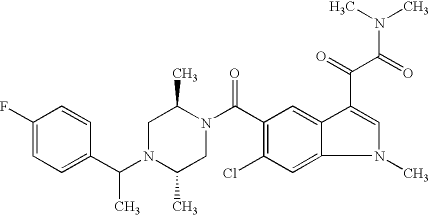 |
| 103 | 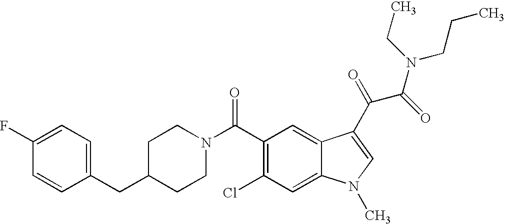 |
| 104 | 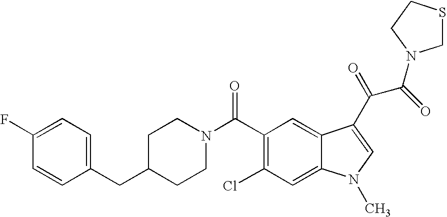 |
| 105 | 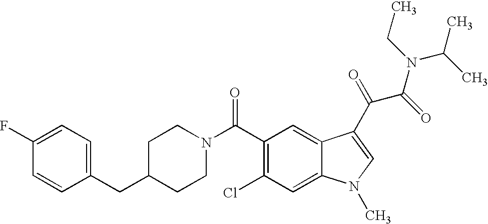 |
| 106 | 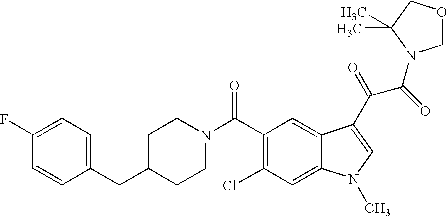 |
| 107 | 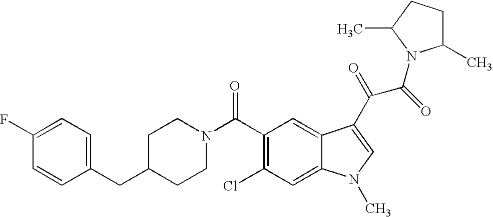 |
| 108 | 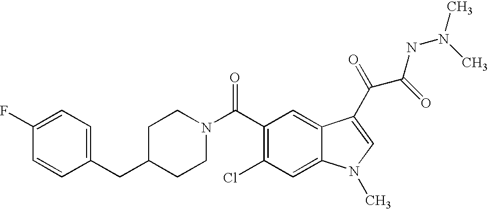 |
| 109 | 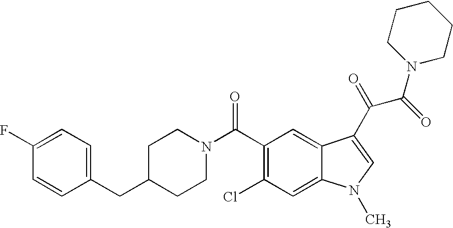 |
| 110 | 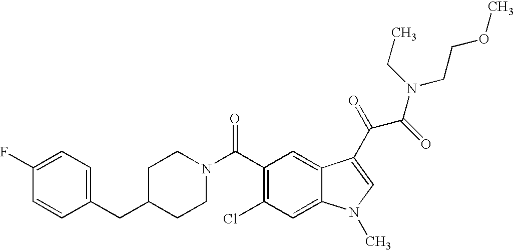 |
| 111 | 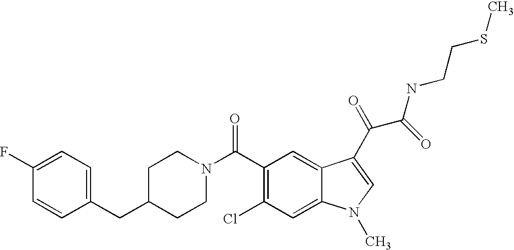 |
| 112 | 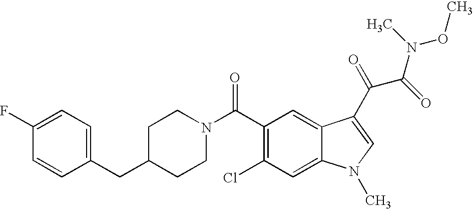 |
| 113 | 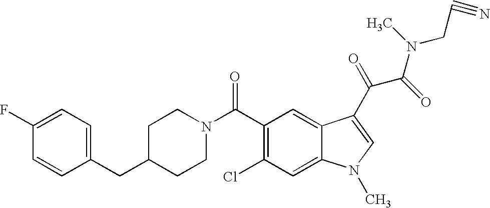 |
| 114 | 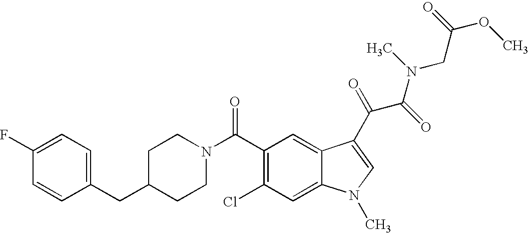 |
| 115 | 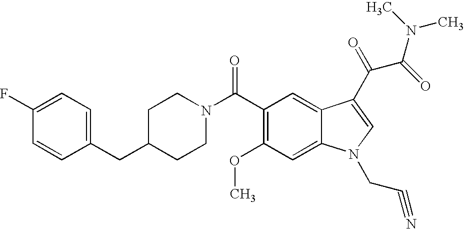 |
| 116 | 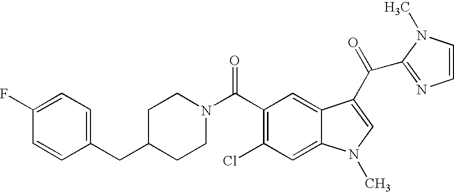 |
| 117 | 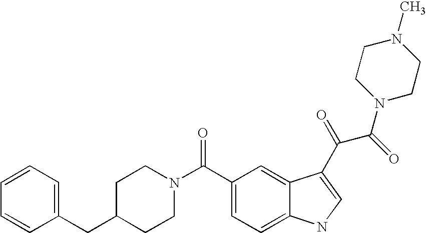 |
| 118 | 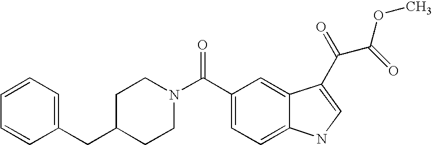 |
| 119 | 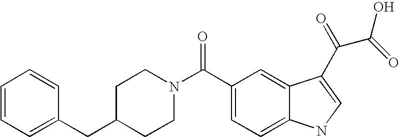 |
| 120 | 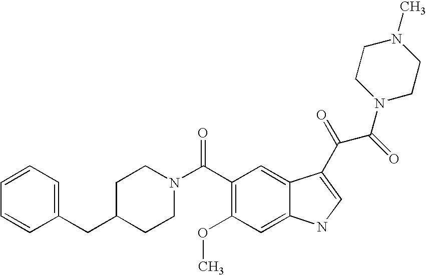 |
| 121 | 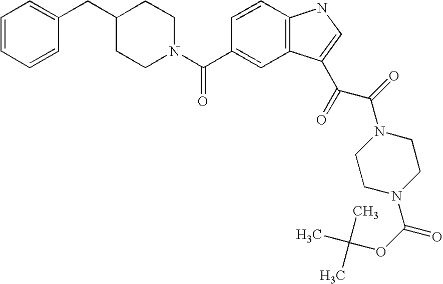 |
| 122 | 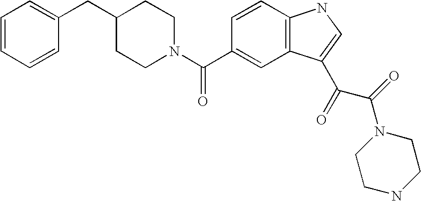 |
| 123 | 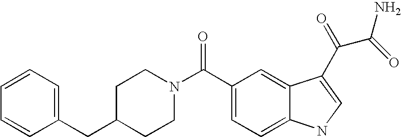 |
| 124 | 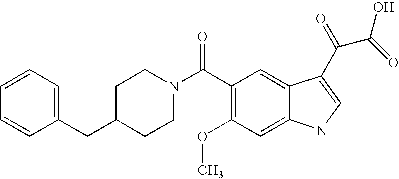 |
| 125 | 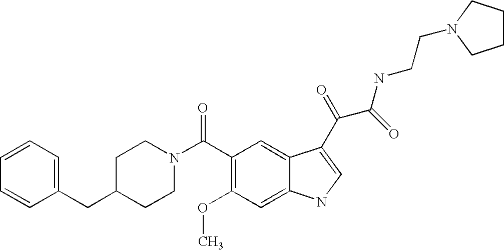 |
| 126 | 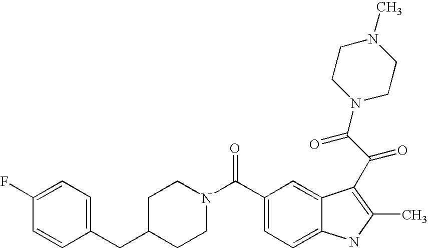 |
| 127 | 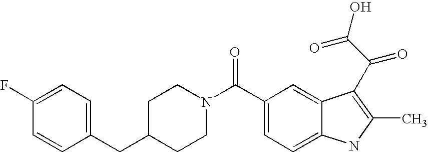 |
| 128 | 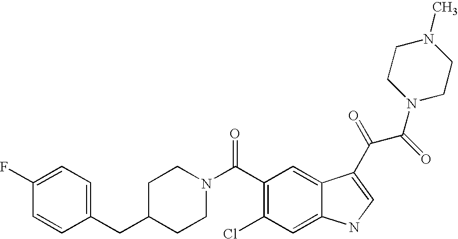 |
| 129 | 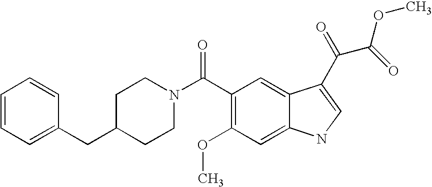 |
| 130 | 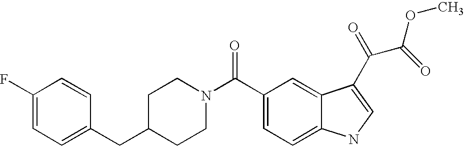 |
| 131 | 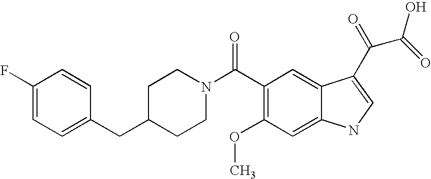 |
| 132 | 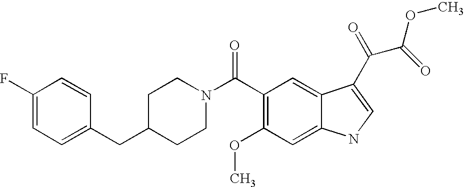 |
| 133 | 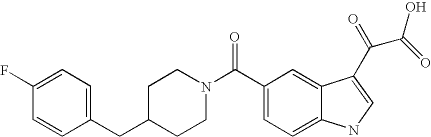 |
| 134 | 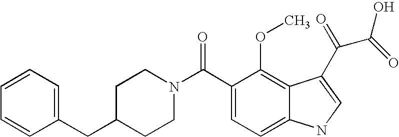 |
| 135 | 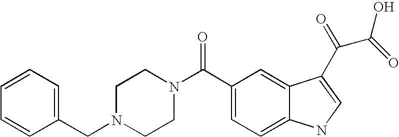 |
| 136 | 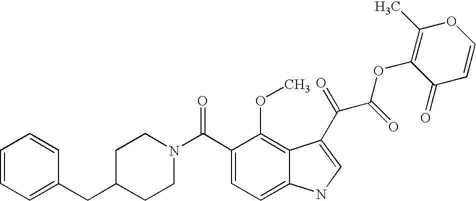 |
| 137 | 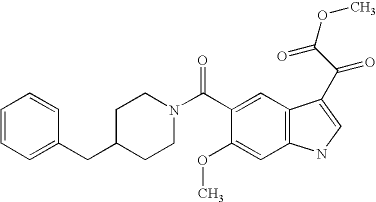 |
| 138 | 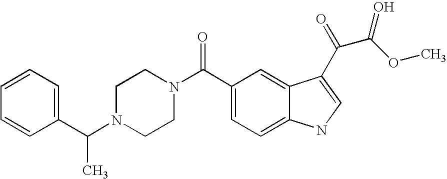 |
| 139 | 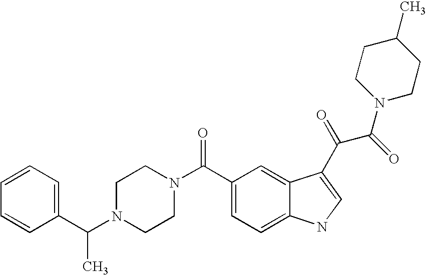 |
| 140 | 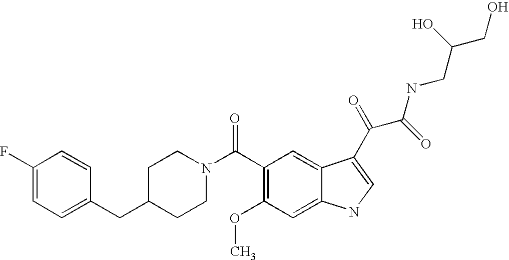 |
| 141 | 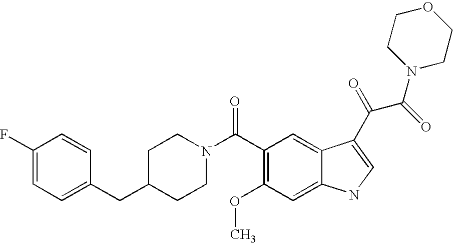 |
| 142 | 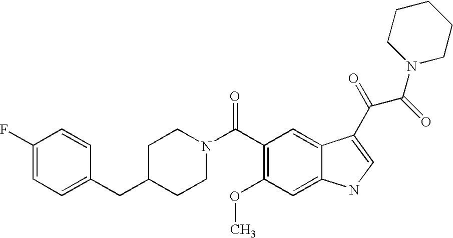 |
| 143 | 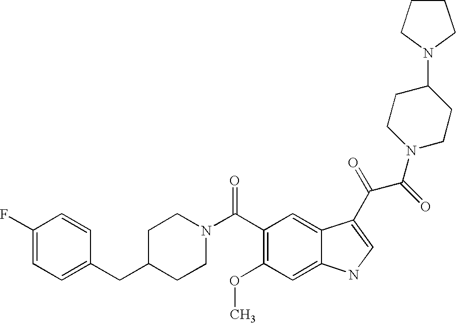 |
| 144 | 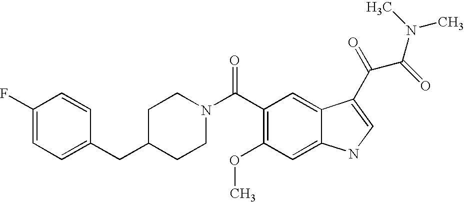 |
| 145 | 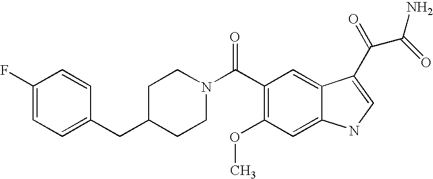 |
| 146 | 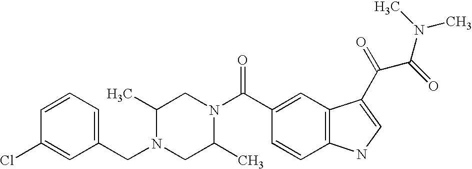 |
| 147 | 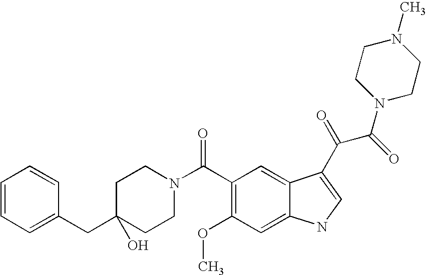 |
| 148 | 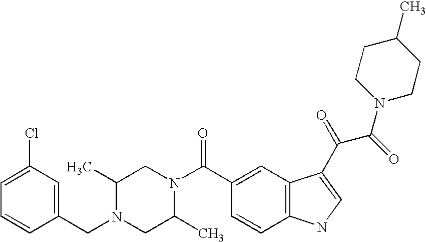 |
| 149 | 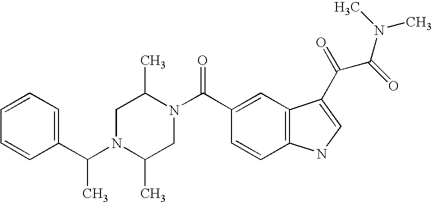 |
| 150 | 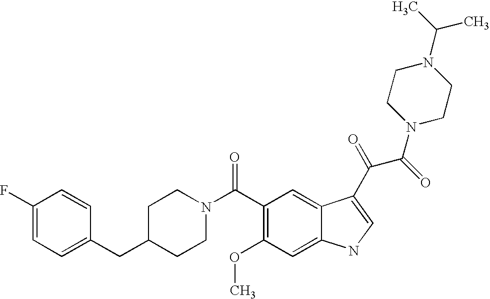 |
| 151 | 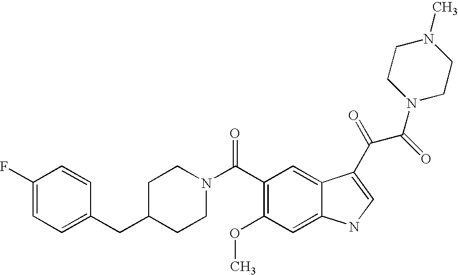 |
| 152 | 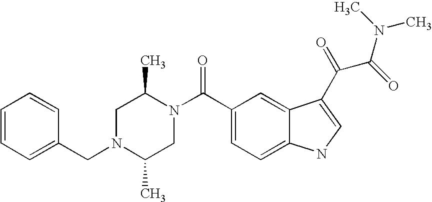 |
| 153 | 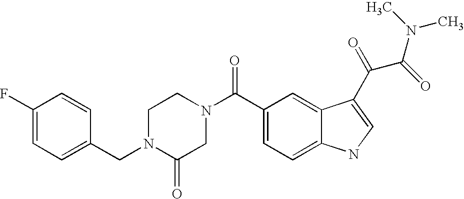 |
| 154 | 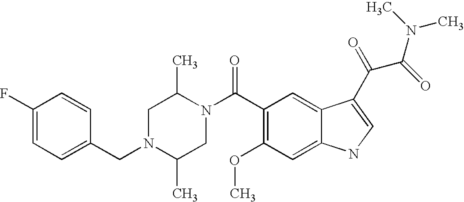 |
| 155 | 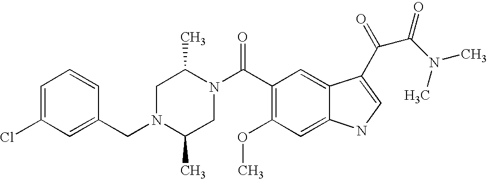 |
| 156 | 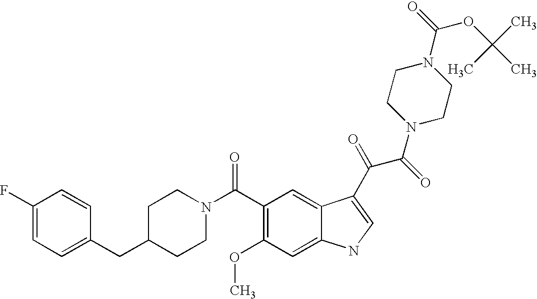 |
| 157 | 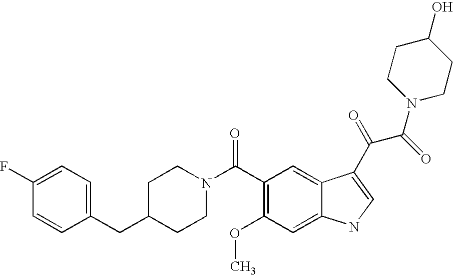 |
| 158 | 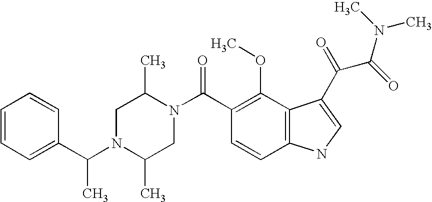 |
| 159 | 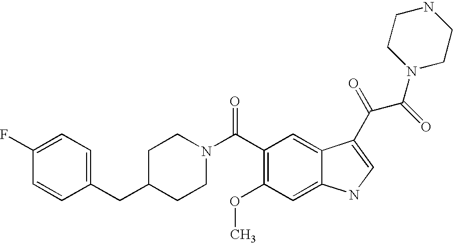 |
| 160 | 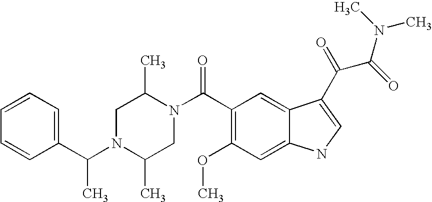 |
| 161 | 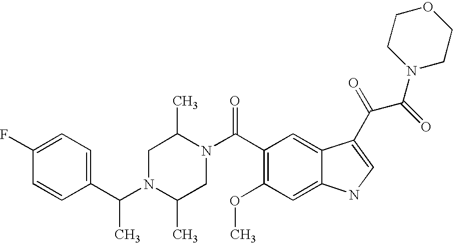 |
| 162 | 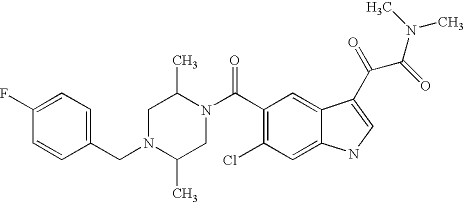 |
| 163 | 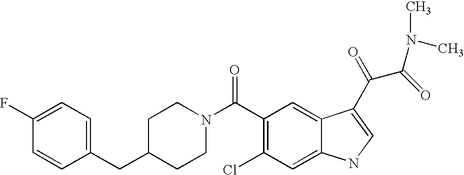 |
| 164 | 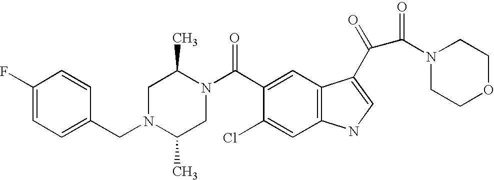 |
| 165 | 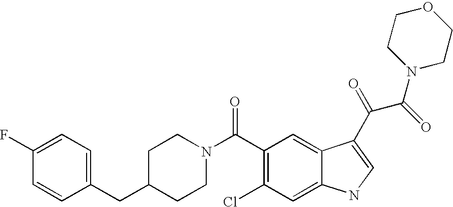 |
| 166 | 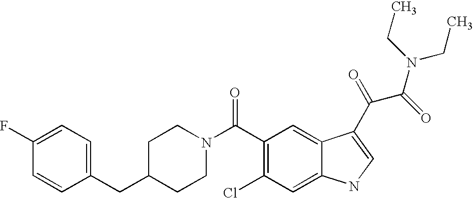 |
| 167 | 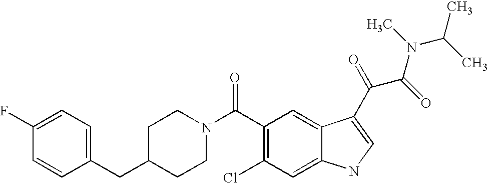 |
| 168 | 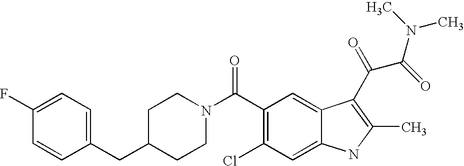 |
| 169 | 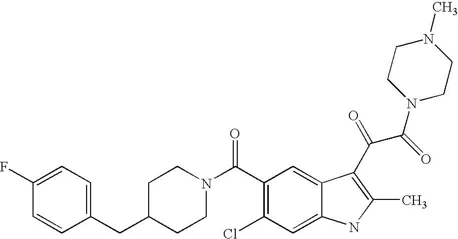 |
| 170 | 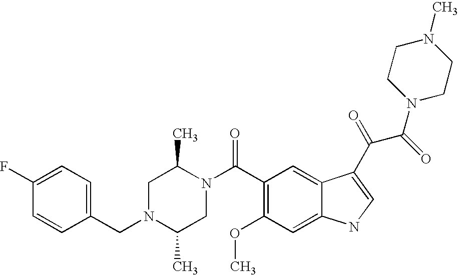 |
| 171 | 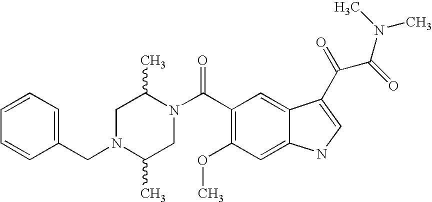 |
| 172 | 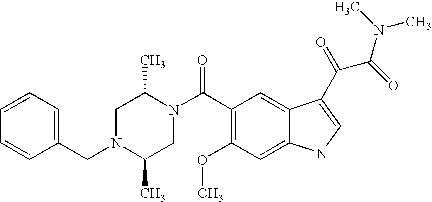 |
| 173 | 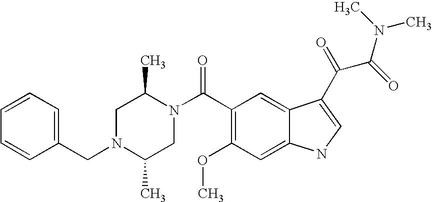 |
| 174 | 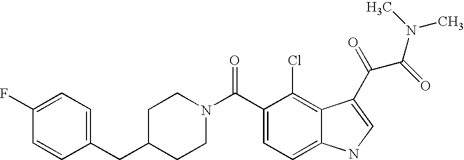 |
| 175 | 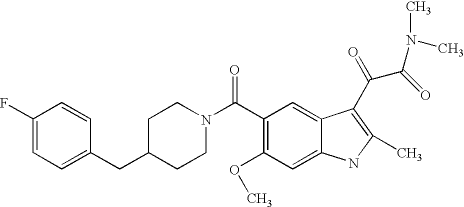 |
| 176 | 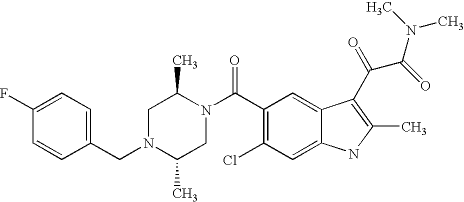 |
| 177 | 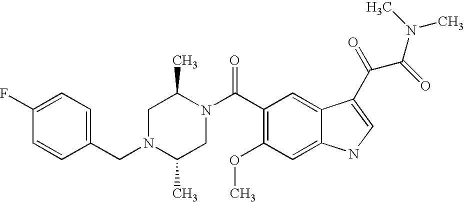 |
| 178 | 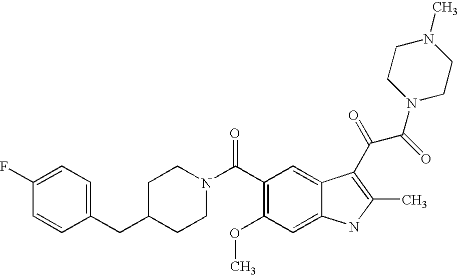 |
| 179 | 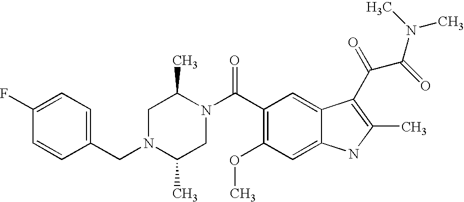 |
| 180 | 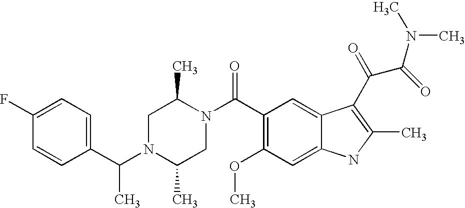 |
| 181 | 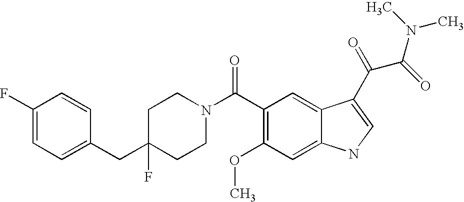 |

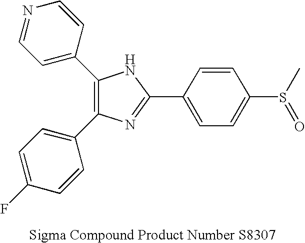

Additional compounds are described in published PCT application WO 96/21452, WO 96/40143, WO 97/25046, WO 97/35856, WO 98/25619, WO 98/56377, WO 98/57966, WO 99/32110, WO 99/32121, WO 99/32463, WO 99/61440, WO 99/64400, WO 00/10563, WO 00/17204, WO 00/19824, WO 00/41698, WO 00/64422, WO 00/71535, WO 01/38324, WO 01/64679, WO 01/66539, and WO 01/66540, each of which is herein incorporated by reference in their entirety.

Further additional compounds useful in the practice of the present invention also include, but are not limited to, the compounds shown in Table 3, below.

TABLE 3

| | Citations, each of which is herein |
|---|---|
| Chemical Structure | incorporated by reference. |
| 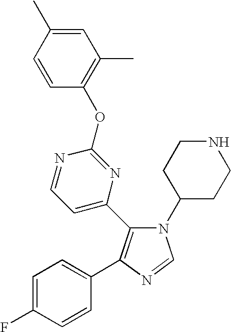 | WO-00166539, WO-00166540, WO-00164679, WO-00138324, WO-00064422, WO-00019824, WO-00010563, WO-09961440, WO-09932121, WO-09857966, WO-09856377, WO-09825619, WO-05756499, WO-09735856, WO-09725046, WO-09640143, WO-09621452; Gallagher, T. F., et. Al., Bioorg. Med. Chem. 5:49 (1997); Adams, J. L., et al., Bioorg. Med. Chem. Lett. 8:3111-3116 (1998) |
| 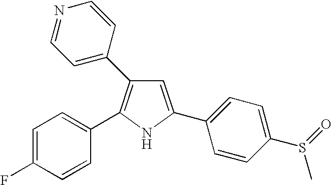 | De Laszlo, S. E., et. Al., Bioorg Med Chem Lett. 8:2698 (1998) |
| 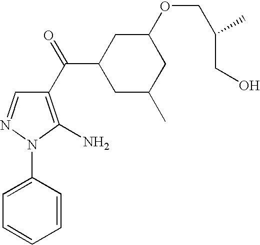 | WO-09957101; Poster presentation at the 5^{th} World Congress on Inflammation, Edinburgh, UK. (2001) |
| 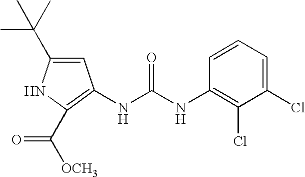 | WO-00041698, WO-09932110, WO-09932463 |
| 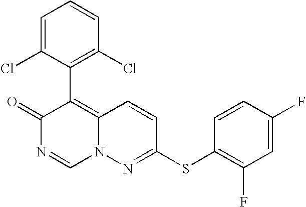 | WO-00017204, WO-09964400 |
| 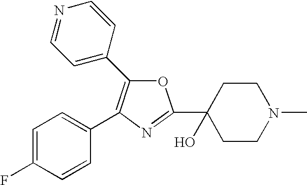 | Revesz. L., et. al., Bioorg Med Chem Lett. 10:1261 (2000) |
| 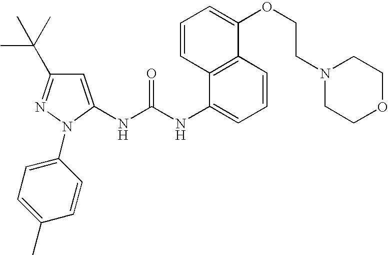 | WO-00207772 |
| 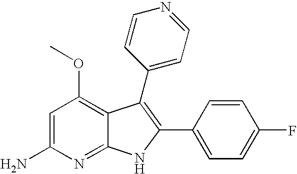 | Fijen, J. W., et al., Clin. Exp. Immunol. 124:16-20 (2001); Wadsworth, S. A., et. al., J. Pharmacol. Expt. Therapeut. 291:680 (1999) |
| 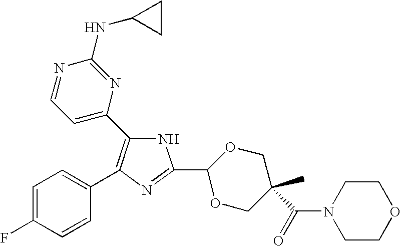 | Collis, A. J., et al.. Bioorg. Med Chem. Lett. 11:693-696 (2001); McLay, L. M., et al., Bioorg Med Chem 9:537-554 (2001) |
| 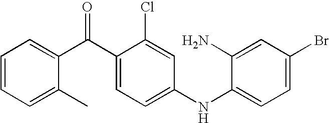 | WO-00110865, WO-00105749 |

Additional guidance regarding p38 MAPK inhibitory compounds is found in U.S. patent application Ser. Nos. 09/575,060 (now U.S. Pat. No. 6,867,209), 10/157,048 (now U.S. Pat. No. 6,864,260), 10/146,703, 10/156,997, and 10/156,996, all of which are hereby incorporated by reference in their entirety. The compounds described above are provided for guidance and exemplary purposes only. It should be understood that any modulator of a p38MAP kinase that plays a role in the genesis and maintenance of the MM disease state is useful for the invention provided that it exhibits adequate activity relative to the targeted protein.

Utility and Administration

The methods and compositions of the invention are successful to treat or ameliorate multiple myeloma in humans. As used herein, “treat” or “treatment” include effecting postponement of development of undesirable conditions and/or reduction in the severity of such symptoms that will or are expected to develop. Treatment includes ameliorating existing symptoms, preventing additional symptoms, ameliorating or preventing the underlying metabolic causes of symptoms, preventing the severity of the condition or reversing the condition, at least partially. Thus, the terms denote that a beneficial result has been conferred on a subject with multiple myeloma.

Treatment generally comprises “administering” to a subject a compound which includes providing the subject compound in a therapeutically effective amount. “Therapeutically effective amount” means the amount of the compound that will treat multiple myeloma by eliciting a favorable response in a cell, tissue, organ, system, in a human. The response may be preventive or therapeutic. The administering may be of the compound per se in a pharmaceutically acceptable composition, or this composition may include combinations with other active ingredients that are suitable to the treatment of this condition. The compounds may be administered in a prodrug form.

The manner of administration and formulation of the compounds useful in the invention and their related compounds will depend on the composition of the compound, the nature of the condition, the severity of the condition, the particular subject to be treated, and the judgment of the practitioner; formulation will also depend on mode of administration. For example, if the compounds are “small molecules,” they might be conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of 5%-95% of the total formulation, but wide variation is permitted depending on the carrier. Suitable carriers include sucrose, pectin, magnesium stearate, lactose, peanut oil, olive oil, water, and the like. This method is preferred if the subject can tolerate oral administration.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically, or in formulation intended to penetrate the skin. These include lotions, creams, ointments and the like which can be formulated by known methods.

The compounds may also be administered by injection, including intravenous, intramuscular, subcutaneous or intraperitoneal injection. Typical formulations for such use are liquid formulations in isotonic vehicles such as Hank's solution or Ringer's solution.

Intravenous administration is preferred for acute conditions; generally in these circumstances, the subject will be hospitalized. The intravenous route avoids any problems with inability to absorb the orally administered drug.

Alternative formulations include nasal sprays, liposomal formulations, slow-release formulations, and the like, as are known in the art.

Any suitable formulation may be used. A compendium of art-known formulations is found in Remington's Pharmaceutical Sciences, latest edition, Mack Publishing Company, Easton, Pa. Reference to this manual is routine in the art.

Thus, the compounds useful in the method of the invention may be administered systemically or locally. For systemic use, the compounds are formulated for parenteral (e.g., intravenous, subcutaneous, intramuscular, intraperitoneal, intranasal or transdermal) or enteral (e.g., oral or rectal) delivery according to conventional methods. Intravenous administration can be by a series of injections or by continuous infusion over an extended period. Administration by injection or other routes of discretely spaced administration can be performed at intervals ranging from weekly to once to three times daily. Alternatively, the compounds may be administered in a cyclical manner (administration of compound; followed by no administration; followed by administration of compound, and the like). Treatment will continue until the desired outcome is achieved. In general, pharmaceutical formulations will include an active ingredient in combination with a pharmaceutically acceptable vehicle, such as saline, buffered saline, 5% dextrose in water, borate-buffered saline containing trace metals or the like. Formulations may further include one or more excipients, preservatives, solubilizers, buffering agents, albumin to prevent protein loss on vial surfaces, lubricants, fillers, stabilizers, etc.

Pharmaceutical compositions can be in the form of sterile, non-pyrogenic liquid solutions or suspensions, coated capsules, suppositories, lyophilized powders, transdermal patches or other forms known in the art.

Biodegradable films or matrices may be used in the invention methods. These include calcium sulfate, tricalcium phosphate, hydroxyapatite, polylactic acid, polyanhydrides, bone or dermal collagen, pure proteins, extracellular matrix components and the like and combinations thereof. Such biodegradable materials may be used in combination with non-biodegradable materials, to provide desired mechanical, cosmetic or tissue or matrix interface properties.

Alternative methods for delivery may include osmotic minipumps; sustained release matrix materials such as electrically charged dextran beads; collagen-based delivery systems, for example; methylcellulose gel systems; alginate-based systems, and the like.

Aqueous suspensions may contain the active ingredient in admixture with pharmacologically acceptable excipients, comprising suspending agents, such as methyl cellulose; and wetting agents, such as lecithin, lysolecithin or long-chain fatty alcohols. The said aqueous suspensions may also contain preservatives, coloring agents, flavoring agents, sweetening agents and the like in accordance with industry standards.

Preparations for topical and local application comprise aerosol sprays, lotions, gels and ointments in pharmaceutically appropriate vehicles which may comprise lower aliphatic alcohols, polyglycols such as glycerol, polyethylene glycol, esters of fatty acids, oils and fats, and silicones. The preparations may further comprise antioxidants, such as ascorbic acid or tocopherol, and preservatives, such as p-hydroxybenzoic acid esters.

Parenteral preparations comprise particularly sterile or sterilized products. Injectable compositions may be provided containing the active compound and any of the well known injectable carriers. These may contain salts for regulating the osmotic pressure.

Liposomes may also be used as a vehicle, prepared from any of the conventional synthetic or natural phospholipid liposome materials including phospholipids from natural sources such as egg, plant or animal sources such as phosphatidylcholine, phosphatidylethanolamine, phosphatidylglycerol, sphingomyelin, phosphatidylserine, or phosphatidylinositol and the like. Synthetic phospholipids may also be used.

The dosages of the compounds of the invention will depend on a number of factors which will vary from subject to subject. However, it is believed that generally, the daily dosage in humans (average weight of 70 kg) will range between 30 mg and 500 mg, preferably between 45 mg and 400 mg, more preferably between 50 mg and 300 mg per day. The dose regimen will vary, however, depending on the compound and formulation selected, the condition of the subject being treated and the judgment of the practitioner. Optimization of dosage, formulation and regimen is routine for practitioners of the art.

The following examples are intended to illustrate but not to limit the invention.

### EXAMPLES

The following examples describe experiments to evaluate the effectiveness of p38 MAPK inhibitors as a treatment for multiple myeloma in a patient in need thereof. Table 2 lists a number of compounds that generally exhibit p38 MAPK activity, preferred embodiments exhibit a relative IC₅₀ value of less than 5 nM in an assay similar to the phosphorylation assay disclosed above (see Kumar). The compounds listed in Table 2 exemplify the compounds generically disclosed herein. Moreover, the data discussed below is representative of the genus of p38 MAPK inhibitors disclosed herein. The results discussed below are thought to be obtainable using any of the p38 MAPK inhibitors disclosed herein. As such, the data provided demonstrates that the genus of p38MAPK inhibitor compounds disclosed herein are useful in the disclosed methods of treating multiple myeloma. The Sigma-Aldrich® under product number S8307 compound is 4-(4-Fluorophenyl)-2-(4-methylsulfinylphenyl)-5-(4-pyridyl)-1H-imidazole, which is known in the literature as a p38 MAPK modulator and commercial available. This compound is available as a positive control in a p38 MAPK inhibition assay.

### Example 1

### p38 Activation in Multiple Myeloma

p38 MAPK is activated via dual phosphorylation by MEKK3 and/or MEKK6. The activated form of p38α MAPK is found in untreated and TNFα-activated MM cells, and in BMSC, using p38α phospho-specific immunodetection. BMSC are obtained from seven different donors (three normal healthy individuals and four MM patients). In the experiments reported here, no differences between BMSCs obtained from patients and healthy individuals were noted. A variety of widely used MM cell lines were also used in the studies described below.

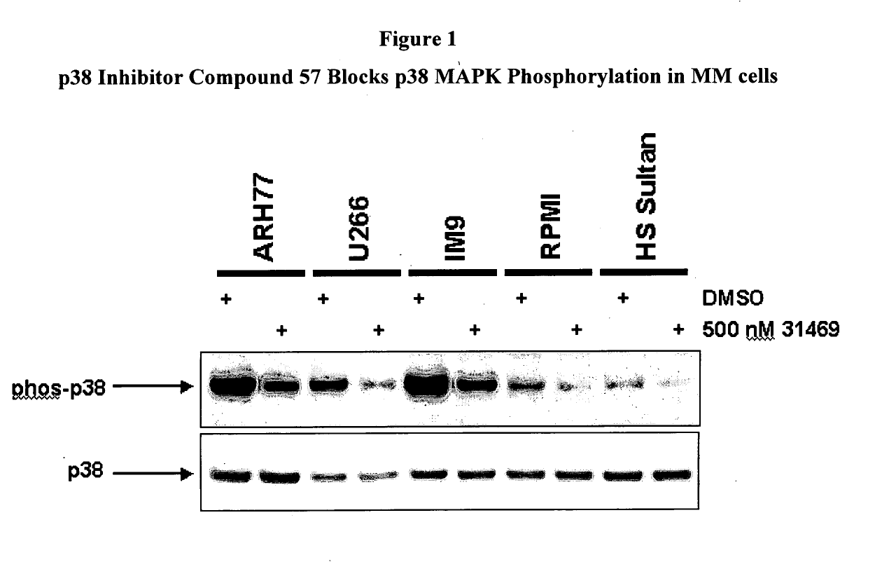

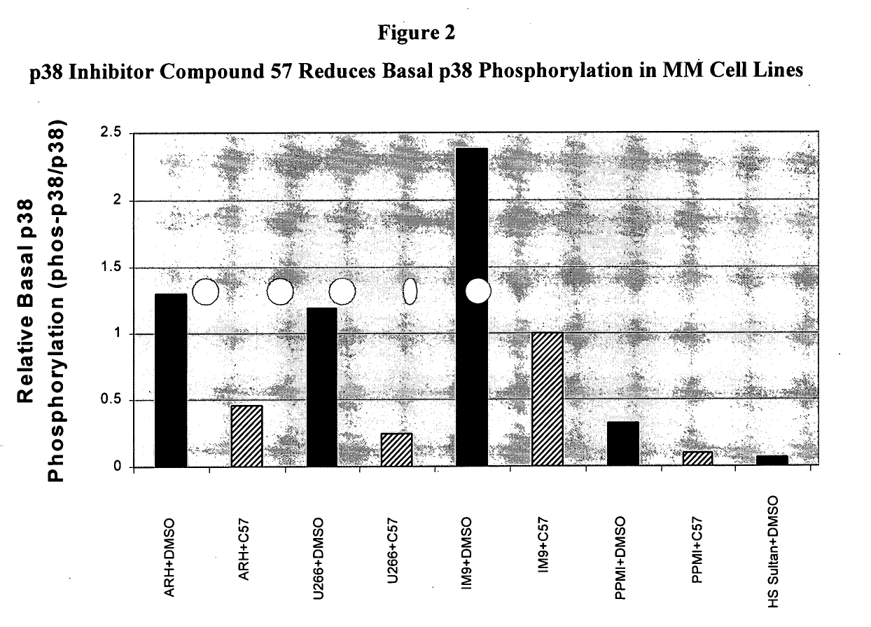

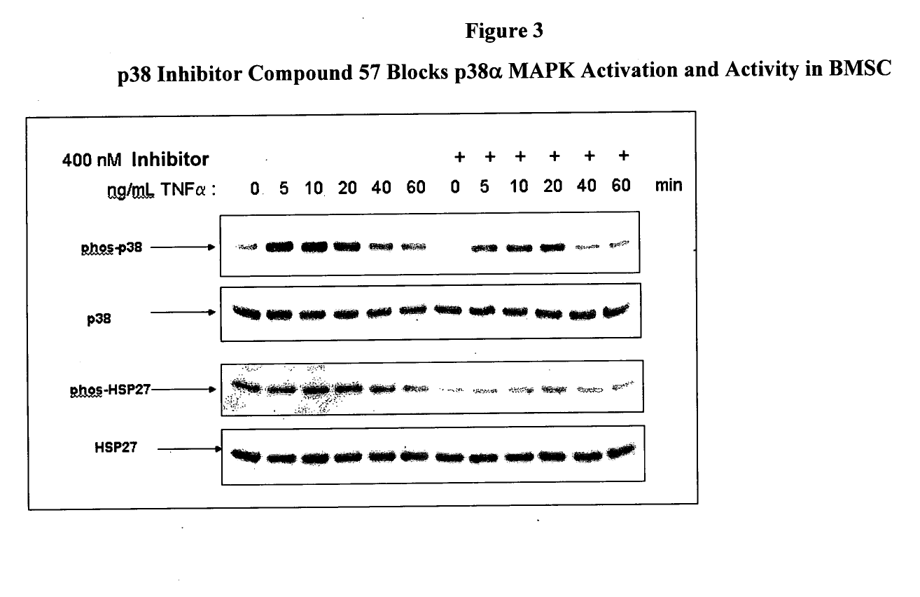

The phosphorylation of p38α MAPK in MM cells was substantially suppressed by the MAPK inhibitor Compound 57 shown in Table 2 (see FIG. 1 and FIG. 2), while the phosphorylation of p38α MAPK in BMSC was partially suppressed (FIG. 3). This inhibitor blocked activity of p38 MAPK, but not the direct activation of the p38 MAPK enzyme or the activity of kinases upstream of p38 MAPK (e.g., MKK3 and MKK6); therefore this cellular effect is presumed to result from disruption of a feedback loop involving p38 MAPK kinase activity. As expected, p38 MAPK activity was fully suppressed by the p38 MAPK inhibitor, shown by immunodetection of p38 MAPK kinase target HSP-27. The Compound 57 blocked phosphorylation of HSP-27 completely in MM cells and in BMSC.

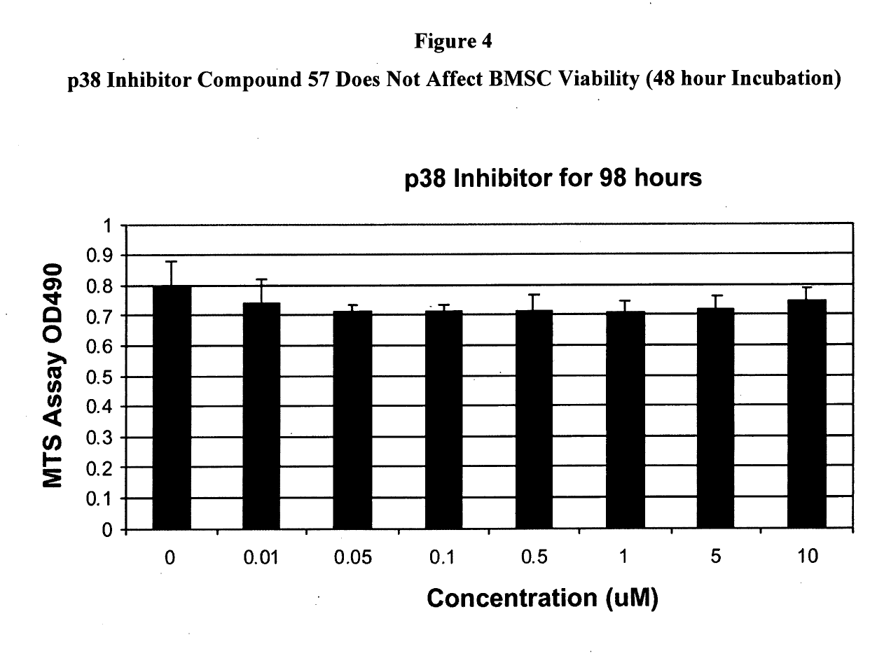

Neither p38 MAPK inhibition nor high concentrations of p38 MAPK inhibitor (tested up to 50-fold excess of active concentration) affected BMSC viability (FIG. 4). Viability was measured using a standard enzymatic assay of respiratory activity, MTS (3-(4,5-dimethylthiazol-2-yl)-5-(3-carboxymethoxyphenyl)-2-(4-sulfophenyl)-2H-tetrazolium). Results obtained with the p38 MAPK inhibitor are in agreement with published results using a different p38α MAPK inhibitor.

### Example 2

### p38α MAPK Inhibition Reduces MM Cell Growth and Proliferation

Phosphorylation of p38α MAPK in MM cells was substantially suppressed by Compound 57, while phosphorylation of p38α MAPK in BMSC was partially suppressed. Neither p38α MAPK inhibition nor high concentrations of Compound 57 (tested up to 50-fold excess of active concentration) affected BMSC viability.

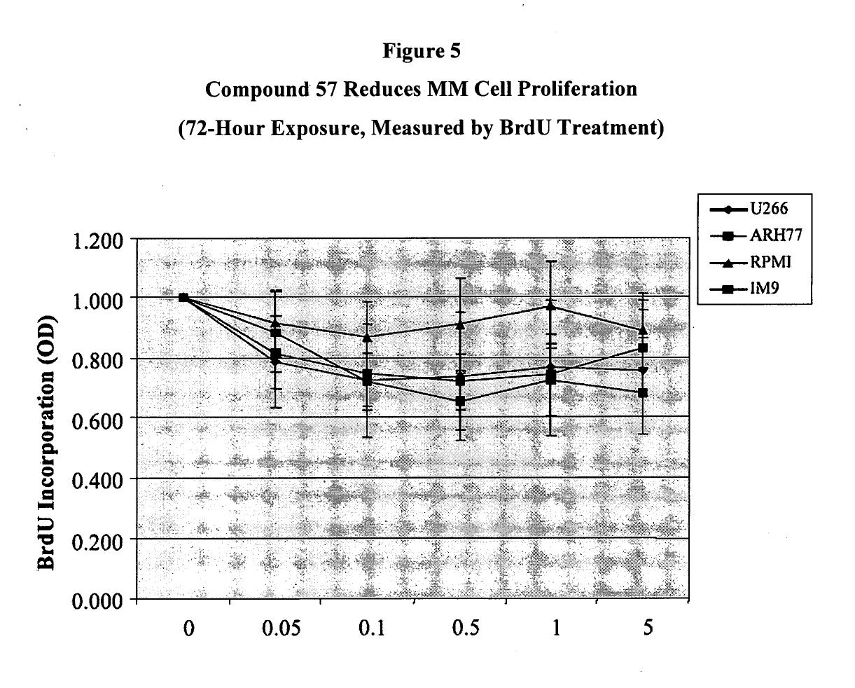

A 72-hour exposure to p38 MAPK inhibitor Compound 57 causes a reduction in proliferation of about 20% in each of four MM cell lines tested (U266B, RPMI, ARH-77, and IM9) (FIG. 5).

### Example 3

### p38α MAPK Inhibition Blocks Cytokine Secretion by Multiple Myeloma and Bone Marrow Stromal Cells

The BM microenvironment contributes to MM cell drug resistance and to osteolytic lesions in adjacent bone. The consequences to patients include tumor growth and disease progression, and bone fractures and pain. Cytokines produced by MM cells, or marrow cells in response to MM cells include TNFα, IL-1β, IL-6, VEGF, and PGE2. Primary among these is IL-6, which promotes MM growth and survival, and resistance to chemotherapy.

In addition to supporting the primary tumor, a number of the cytokines produced by MM and marrow cells promote development and activation of osteoclasts. These cells display an inappropriately high level of activity in MM and are responsible for the development of osteolytic lesions. Factors thought to play a primary role in this aspect of MM include IL-11, MIP-1 and RANKL.

p38 MAPK controls secretion of these factors in-vitro in systems representing the BM milieu. To demonstrate this with p38 MAPK inhibitor Compound 57 of Table 2, various models were used including MM cells cultured alone, primary human BMSC cultured alone, MM cells co-cultured with BMSC, and BMSC cultured in the presence of cytokines which are elevated in MM.

TNF-α

Although circulating levels of TNFα are elevated in MM patients, and TNFα is present in MM marrow, secretion of this major inflammatory cytokine is not detected in BMSC or MM cultures under basal conditions or using the stimulus set that induced other MM-relevant signaling molecules. Macrophages are major producers of TNFα and are likely to be a primary source of TNFα in MM.

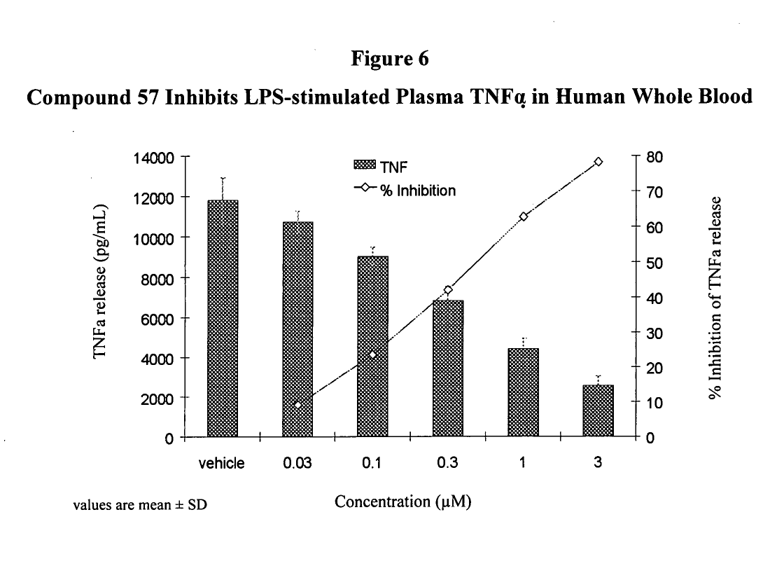

TNFα plays a role in controlling the production of numerous MM-relevant cytokines in marrow systems and is elevated in MM serum and marrow samples. Exposure of whole blood to Compound 57 caused dose-dependent reduction in LPS-induced TNF-α secretion (FIG. 6).

With regard to inflammatory prostanoids, p38 MAPK inhibitor Compound 57 inhibited LPS-induced COX-2 mRNA. PGE2 levels were also reduced in LPS-induced human whole blood treated with p38 MAPK inhibitor Compound 57, consistent with reduced COX-2 protein and mRNA levels. By virtue of its impact on COX-2 protein synthesis, the p38 MAPK inhibitor Compound 57 blocked PGE2 synthesis but did not directly inhibit the activity of the purified COX-2 enzyme, nor did it inhibit the activity of a platelet-derived preparation of COX-1. The results with p38 MAPK inhibitor Compound 57 are consistent with a literature report demonstrating that p38 MAP kinase mediates COX-2 mRNA induction.

Interleukin-1β

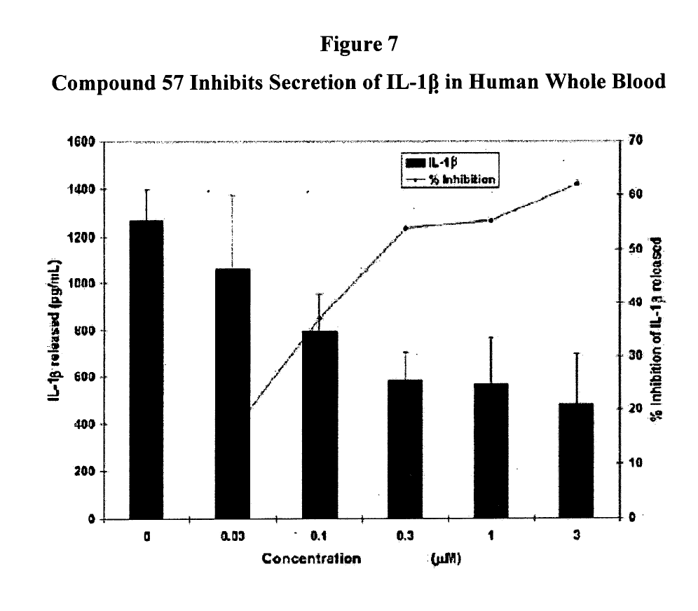

IL-1β illustrates the importance of amplification loops in myeloma-induced cytokine stimulation. IL-1, plays a well-documented role in the induction of multiple cytokines in a “cytokine network” of feedback loops, including strong induction of IL-6. The ability of IL-1 to increase mRNA expression and induce secretion of the MM-relevant cytokines from MM/BMSC cultures is evidence of this role. Exposure of human whole blood (undiluted) to Compound 57 caused a dose-dependent reduction in LPS-induced IL-1β release (FIG. 7).

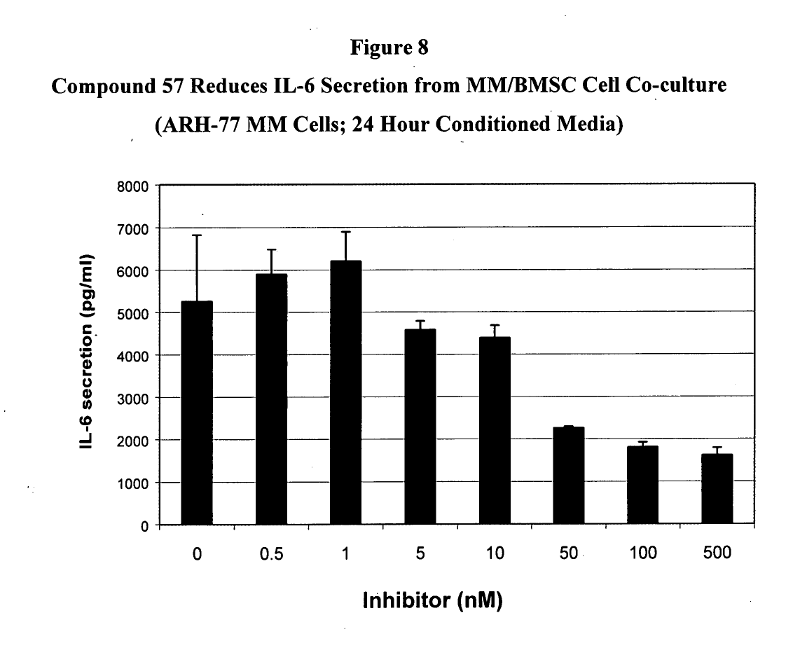

Interleukin-6
p38α MAPK inhibition potently blocked IL-6 production and secretion MM/BMSC cell co-cultures (FIG. 8), and by MM and BMSC cells. The observed reduction resulted, at least in part, from inhibition of IL-6 mRNA production (data not shown).

Interleukin-11 (IL-11)

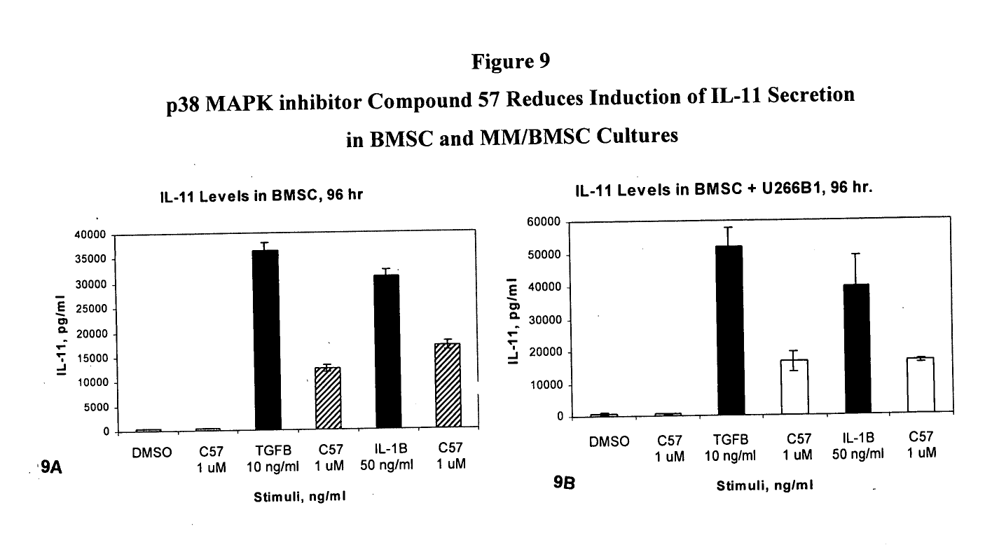

IL-11 elicits pro-fibrotic responses and promotes osteoclast-driven bone resorption. In both BMSC and MM/BMSC cultures, 100 nM concentrations of Compound 57 blocked IL-1 and TGFβ powerfully induction of IL-11 at the mRNA and protein levels (FIG. 9).

Vascular Endothelial Growth Factor (VEGF)

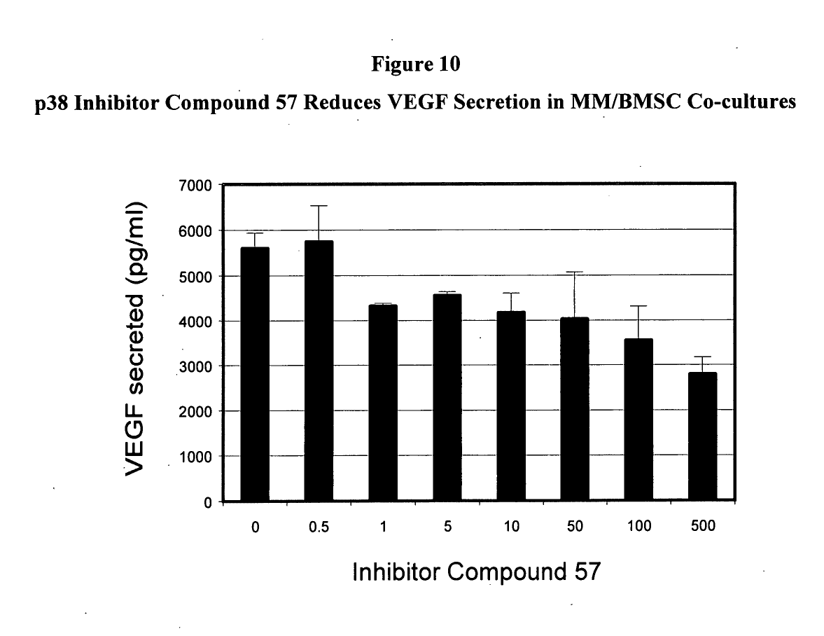

In addition to promoting vascularization, VEGF is a growth-supporting factor for MM cells. Neovascularization of MM tumors in the marrow allows growth beyond diffusion limitations for gas and nutrient exchange. VEGF is secreted from MM contact-activated BMSC, and this secretion is inhibited by p38 MAPK inhibitor Compound 4 (FIG. 10).

### Example 7

### p38 MAPK Inhibition in the Presence of Chemotherapeutic Agents and Potential Effects on MM Cells

p38 MAPK activity is necessary for production of factors such as IL-6, and therefore contributes to MM cell survival, even in the presence of chemotherapeutic agents. For example, a p38-mediated stress response in MM cells may invoke protective mechanisms, and in BMSC may induce secretion of MM survival factors such as IL-6. In fact, the unusual chemoresistance of MM cells compared to other B cell malignancies is thought to be due in part to IL-6-mediated support of MM cells.

Inhibition of p38 MAPK might enhance the effects of conventional MM chemotherapeutic treatment through one or both of two mechanisms: 1) by down-regulating the activity of one or more of these factors; 2) through an overlap of mechanisms of action with some of the newer therapeutic agents.

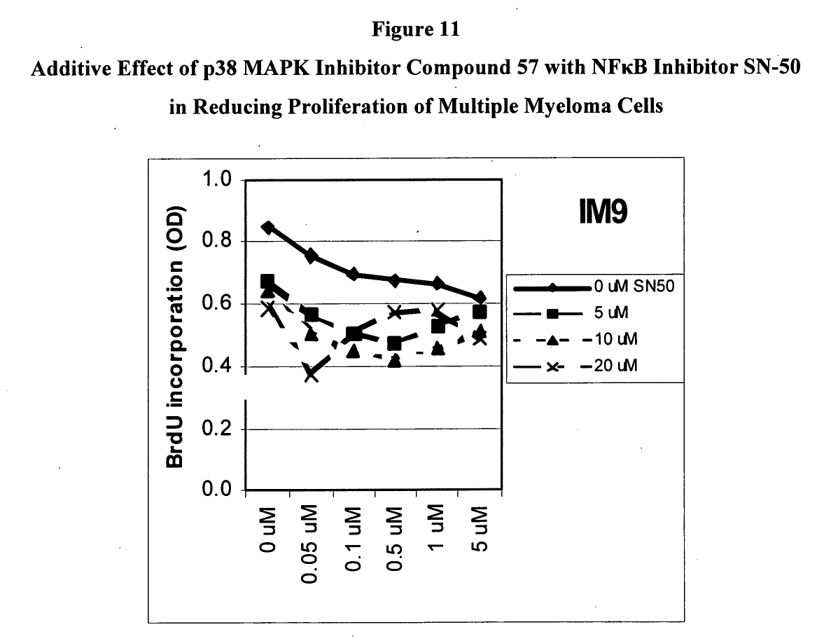

Co-exposure to the p38 MAPK inhibitor Compound 57 enhances the reduction of MM cell viability and proliferation caused by SN-50-induced-NFκB inhibition (FIG. 11). SN-50 is a cell-permeable specific inhibitor of NF-kappaB nuclear translocation and activity. Nuclear factor-kappaB has been reported to confer significant survival potential in a variety of tumors, including MM cells.

These interactions suggest potential benefits of co-treatment regimes in MM. Since dose limiting toxicities are observed with many treatments, the ability to achieve equivalent efficacy at lower doses, or to allow tolerance of a higher dose in order to further increase response, has clear potential for therapeutic benefit.

### Example 8

### p38 MAPK Inhibition Blocks Hsp27 Phosphorylation in MM Cells

Hsp27 has been implicated as an important factor in the development of drug resistance by MM cells. Therefore, Hsp27 is an attractive therapeutic target. Because Hsp27 is downstream of p38 MAP kinase in a signaling cascade (p38→MAPKAPK-2→Hsp27), an attempt is made to determine whether Hsp27 phosphorylation can be inhibited with various p38 MAPK inhibitors of Table 2 (i.e., Compound 57), that are potent inhibitors of p38 MAPK.

For these studies, U266, IM9 and RPMI8226 cells are incubated with DMSO (−) or with 0.5 μM of a disclosed p38 MAPK inhibitor (+) for 1 hour and cell lysates are immunoblotted with antibodies to phospho-p38 and p38 MAP kinase by Western analysis. U266B1 and RPMI8226 are MM cell lines, and IM9 is an Epstein Barr Virus (EBV)-transformed B cell line with characteristics of MM cells. All can be obtained from American Type Culture Collection (ATCC; Rockville, Md.). All cell lines are maintained in RPMI-1640 (ATCC), supplemented with 10% fetal bovine serum (Hyclone; Logan, Utah), 100 units/ml of penicillin, 100 μg/ml streptomycin and 2 mM L-glutamine (Life Technologies, Inc.; Grand Island, N.Y.). BMSC (Cambrex) are maintained in Myelocult H5100 supplemented with 10⁻⁶ M hydrocortisone (Stem Cell Technologies; Vancouver, B.C.), 100 units/ml of penicillin and 100 μg/ml streptomycin. MM cells are usually seeded at a density of 3×10⁴ cells/well in 96-well culture plates. For MM co-cultures, BMSC are first seeded at 1.2×10⁴ cells/well in a 96-well plate in Myelocult/hydrocortisone medium for 24 hours prior to the addition of MM cells.

Total cell lysates are immunoprecipitated with anti-MAPKAPK-2 antibody and subjected to in vitro kinase assays using purified GST-Hsp27 as substrate. To examine Hsp27 phosphorylation in MM and transformed B cells, U266, IM9 and RPMI8226 cells are incubated either with DMSO or with 0.5 μM of a p38 MAPK inhibitor and cell lysates are immunoblotted with antibodies to phospho-Hsp27 (Ser 82) and Hsp27.

In additional experiments, the p38 MAPK inhibitors are tested for their ability to inhibit Hsp27 phosphorylation. U266, IM9 and RPMI8226 cells are incubated with 0.5 μM of the p38 MAPK inhibitor for 1 hour and Hsp27 proteins are immunoprecipitated with agarose-conjugated Hsp27 antibody, followed by immunoblotting with anti-phospho Hsp27 (Ser 78) antibody.

Specific antibodies to p38 MAP kinase are from Santa Cruz Biotechnology (Santa Cruz, Calif.). Antibodies to phospho-p38 MAP kinase (T180/Y182) and phospho-Hsp27 (S82) are from Cell Signaling (Beverly, Mass.). Antibodies to Hsp27 and phospho-Hsp27 (S78) are from Upstate Biotechnology (Lake Placid, N.Y.). Anti-MAPKAPK-2 is from StressGen (San Diego, Calif.) while anti-GAPDH is from Biogenesis Ltd. (Poole, UK).

Initially, the phosphorylation state of p38 in the MM cell lines U266 and RPMI8226 is examined as well as the EBV-transformed B cell line IM9, which has MM like characteristics. Interestingly, it is observed that these cells have high basal p38 phosphorylation levels and that stimulating these cells with TNFα does not lead to any appreciable increase in p38 phosphorylation or activation. Addition of the p38 MAPK inhibitor Compound 57 substantially suppresses p38 phosphorylation in all three cell lines. The p38 MAPK inhibitor blocks the catalytic activity of p38α but not the ability of p38 to act as a substrate for upstream MAPKKs (MKK3 and MKK6) nor do they indirectly inhibit MKK3 or MKK6 activity. Thus, the reduced p38 phosphorylation may be the result of a disrupted feedback loop involving p38 kinase activity and is manifested in blocked autophosphorylation. Consistent with inhibiting p38 kinase activity, Compound 57 suppresses the activity of downstream substrate MAPKAPK-2, as measured in an in vitro kinase assay. Finally, Compound 57 inhibits Hsp27 phosphorylation, as determined by direct examination of cell lysates or in immunoprecipitation assays. While Compound 57 inhibits Hsp27 phosphorylation, it does not affect total Hsp27 levels in these cells even after prolonged incubation with these inhibitors. These outcomes should demonstrate that the p38 MAPK inhibitors described herein effectively inhibit the components of the p38 MAP kinase pathway.

### Example 9

### A p38-alpha MAP Kinase Inhibitor Inhibits Human Myeloma Cell Growth In Vivo

The p38 MAPK signaling pathway plays an important role in different pathological conditions and specific inhibitors of p38 alpha and beta MAPK block production of the major inflammatory cytokines. Human-multiple myeloma (MM) is an incurable neoplasm of terminally differentiated B cells and MM disease progression is affected by immunoregulatory elements such as IL-6 and other cytokines. Recent literature indicates that an important function of p38 MAP kinase is the generation of signals of critical value to the control of normal and malignant hematopoiesis by cytokines and growth factors. The effect of the p38 alpha MAP kinase specific inhibitor Compound 57 on human myeloma (RPMI8266) tumor growth is studied in an immunodeficient beige-nude-xid mouse xenograft plasmacytoma model. Treatment (90 mg/kg/bid by oral gavage) is initiated in mice with palpable tumor size (˜200 mm³) as judged by tumor volume. In mice with palpable tumors, 21 days of Compound 57 treatment significantly reduces tumor volume. When Compound 57 is initiated in mice with pronounced tumor size (˜800 mm³), a significant reduction in tumor volume occurs. Histological assessment at the end of dosing from animals administered the exemplary compound or vehicle demonstrates a significant reduction in tumor volume and number of neoformed microvessels in the p38 MAPK Compound 57 treatment groups. Compound 57 also significantly reduces HSP27 and p38 expressions in tumor cells.

### Example 10

### Inhibitors of p38α MAPK for Treating a Subject with Multiple Myeloma

A patient is diagnosed with multiple myeloma. The patient presents with MM cells with rapid growth rates, which displace osteoblasts, and disrupting the balance of bone creation and destruction. A number of MM-related cytokines, such as IL-6, VEGF, IL-11, and PGE-2 are detectable. A therapeutic amount of p38 MAPK inhibitor Compound 57 is administered. MM cell growth is inhibited and MM-related cytokine product is reduced.

### Example 11

### Inhibitors of p38α MAPK and Chemotherapy for Treating a Subject with Multiple Myeloma

A patient is diagnosed with multiple myeloma. The patient presents with MM cells with rapid growth rates, which displace osteoblasts, and disrupting the balance of bone creation and destruction. A number of MM-related cytokines, such as IL-6, VEGF, IL-11, and PGE-2 are detectable. A therapeutic amount of p38 MAPK inhibitor Compound 57 in combination with an apoptosis promoting agent is administered. MM cell growth is inhibited and MM-related cytokine product is reduced.

What is claimed is:

1. A method to treat multiple myeloma in a subject, comprising:
administering to a subject in need of such treatment a therapeutically effective amount of a p38 inhibitor, whereby a symptom associated with multiple myeloma is ameliorated.

2. The method of claim 1, wherein the p38 inhibitor is of the formula: 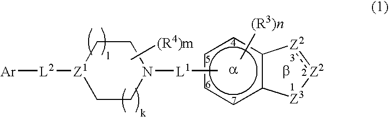
and the pharmaceutically acceptable salts thereof, or a pharmaceutical composition thereof, wherein [symbol]
represents a single or double bond;
one Z² is CA or CR⁶A and the other is CR¹ or CR¹₂, wherein each R¹ is independently hydrogen is alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof;
R⁶ is H, alkyl, alkenyl, alkynyl, aryl, arylalkyl, acyl, aroyl, heteroaryl, heteroalkyl, heteroalkenyl, heteroalkynyl, heteroalkylaryl, SOR, SO₂R, RCO, COOR, alkyl-COR, SO₃R, CONR₂, SO₂NR₂, CN, CF₃, or R₃Si wherein each R is independently H, alkyl, alkenyl or aryl or the heteroforms thereof;
A is —Wᵢ—COXⱼY wherein Y is COR² wherein R² is hydrogen, straight or branched chain alkyl, alkenyl, alkynyl, aryl, arylalkyl, heteroalkyl, heteroaryl, or heteroarylalkyl, each optionally substituted with halo, alkyl, SR, OR, NR₂, OCOR, NRCOR, NRCONR₂, NRSO₂R, NRSO₂NR₂, OCONR₂, CN, COOR, CONR₂, COR, or R₃Si wherein each R is independently H, alkyl, alkenyl or aryl or the heteroforms thereof, or
wherein R² is OR, NR₂, NRCONR₂, OCONR₂, NRSO₂NR₂, heteroarylalkyl, COOR, NRNR₂, heteroaryl, heteroaryloxy, heteroaryl-NR, or NROR wherein each R is independently H, alkyl, alkenyl or aryl or the heteroforms thereof, and wherein two R attached to the same N atom may form a 3-8 member ring and wherein said ring may further be substituted by alkyl, alkenyl, alkynyl, aryl, arylalkyl, heteroaryl, heteroalkyl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroforms thereof wherein two R attached to the same atom may form a 3-8 member ring, optionally substituted as above defined, and
each of W and X is substituted or unsubstituted alkylene or alkenylene, each of 2-6 Å, or
Y is tetrazole; 1,2,3-triazole; 1,2,4-triazole; or imidazole;
each of i and j is independently 0 or 1;
Z³ is NR⁷ or 0;
R⁷ is H or is optionally substituted alkyl, alkenyl, alkynyl, aryl, arylalkyl, acyl, aroyl, heteroaryl, heteroalkyl, heteroalkenyl, heteroalkynyl, or heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof;
each R³ is independently halo, alkyl, heteroalkyl, OCOR, OR, NRCOR, SR, or NR₂, wherein R is H, alkyl or aryl or the heteroforms thereof,
n is 0-3;
L¹ is CO, SO₂ or alkylene (1-4C);
L² is alkylene (1-4C) or alkenylene (2-4C) optionally substituted with one or two moieties selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, and R₃Si, wherein each R is independently H, alkyl, alkenyl or aryl or the heteroforms thereof, and wherein two substituents on L² can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety;
each R⁴ is independently selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof;
m is 0-4;
Z¹ is CR⁵ or N wherein R⁵ is hydrogen or OR, NR₂, SR or halo, wherein each R is independently H, alkyl, alkenyl or aryl or the heteroforms thereof;
each of 1 and k is an integer from 0-2 wherein the sum of 1 and k is 0-3;
Ar is an aryl group substituted with 0-5 noninterfering substituents selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and wherein two of said optional substituents on adjacent positions can be joined to form a fused, optionally substituted aromatic or nonaromatic, saturated or unsaturated ring which contains 3-8 members.

3. The method of claim 1, wherein the symptom comprises a rate of MM cell growth which is reduced as compared to the MM cell growth rate of untreated MM cells.

4. The method of claim 1, wherein the symptom comprises production of a MM-related cytokine, which is reduced.

5. The method of claim 4, wherein the MM-related cytokine is selected from the group consisting of IL-6, VEGF, IL-11, and PGE-2.

6. A method of inhibiting cytokine secretion from multiple myeloma (MM) cells, comprising:
providing a p38 MAP kinase inhibitor to a MM cell, whereby the secretion rate of a MM-related cytokine is reduced as compared to the secretion rate of a MM-related cytokine of untreated MM cells.

7. The method of claim 6, wherein the MM cell resides in a subject suffering from MM.

8. The method of claim 6, wherein the MM-related cytokine is selected from the group consisting of IL-6, VEGF, IL-11, and PGE-2.

9. The method of claim 6, wherein the p38 inhibitor is of the formula: 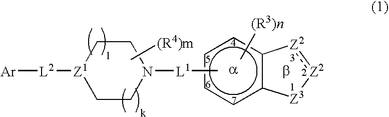
and the pharmaceutically acceptable salts thereof, or a pharmaceutical composition thereof, wherein [symbol]
represents a single or double bond;
one Z² is CA or CR⁶A and the other is CR¹ or CR¹₂, wherein each R¹ is independently hydrogen is alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof;
R⁶ is H, alkyl, alkenyl, alkynyl, aryl, arylalkyl, acyl, aroyl, heteroaryl, heteroalkyl, heteroalkenyl, heteroalkynyl, heteroalkylaryl, SOR, SO₂R, RCO, COOR, alkyl-COR, SO₃R, CONR₂, SO₂NR₂, CN, CF₃, or R₃Si wherein each R is independently H, alkyl, alkenyl or aryl or the heteroforms thereof;
A is —Wᵢ—COXⱼY wherein Y is COR² wherein R² is hydrogen, straight or branched chain alkyl, alkenyl, alkynyl, aryl, arylalkyl, heteroalkyl, heteroaryl, or heteroarylalkyl, each optionally substituted with halo, alkyl, SR, OR, NR₂, OCOR, NRCOR, NRCONR₂, NRSO₂R, NRSO₂NR₂, OCONR₂, CN, COOR, CONR₂, COR, or R₃Si wherein each R is independently H, alkyl, alkenyl or aryl or the heteroforms thereof, or
wherein R² is OR, NR₂, NRCONR₂, OCONR₂, NRSO₂NR₂, heteroarylalkyl, COOR, NRNR₂, heteroaryl, heteroaryloxy, heteroaryl-NR, or NROR wherein each R is independently H, alkyl, alkenyl or aryl or the heteroforms thereof, and wherein two R attached to the same N atom may form a 3-8 member ring and wherein said ring may further be substituted by alkyl, alkenyl, alkynyl, aryl, arylalkyl, heteroaryl, heteroalkyl, heteroarylalkyl, each optionally substituted with halo, SR, OR, NR₂, OCOR, NRCOR, NRCONR₂, NRSO₂R, NRSO₂NR₂, OCONR₂, or R₃Si wherein each R is independently H, alkyl, alkenyl or aryl or the heteroforms thereof wherein two R attached to the same atom may form a 3-8 member ring, optionally substituted as above defined, and
each of W and X is substituted or unsubstituted alkylene or alkenylene, each of 2-6 Å, or
Y is tetrazole; 1,2,3-triazole; 1,2,4-triazole; or imidazole;
each of i and j is independently 0 or 1;
Z³ is NR⁷ or O;
R⁷ is H or is optionally substituted alkyl, alkenyl, alkynyl, aryl, arylalkyl, acyl, aroyl, heteroaryl, heteroalkyl, heteroalkenyl, heteroalkynyl, or heteroalkylaryl, or is SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, alkyl-CONR₂, or R₃Si, wherein each R is independently H, alkyl, alkenyl or aryl or heteroforms thereof;
each R³ is independently halo, alkyl, heteroalkyl, OCOR, OR, NRCOR, SR, or NR₂, wherein R is H, alkyl or aryl or the heteroforms thereof;
n is 0-3;
L¹ is CO, SO₂ or alkylene (1-4C);
L² is alkylene (1-4C) or alkenylene (2-4C) optionally substituted with one or two moieties selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, and R₃Si, wherein each R is independently H, alkyl, alkenyl or aryl or the heteroforms thereof, and wherein two substituents on L can be joined to form a non-aromatic saturated or unsaturated ring that includes 0-3 heteroatoms which are O, S and/or N and which contains 3 to 8 members or said two substituents can be joined to form a carbonyl moiety or an oxime, oximeether, oximeester or ketal of said carbonyl moiety;
each R⁴ is independently selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and two of R⁴ on adjacent positions can be joined to form a fused, optionally substituted aromatic or nonaromatic, saturated or unsaturated ring which contains 3-8 members, or R⁴ is ═O or an oxime, oximeether, oximeester or ketal thereof;
m is 0-4;
Z¹ is CR⁵ or N wherein R⁵ is hydrogen or OR, NR₂, SR or halo, wherein each R is independently H, alkyl, alkenyl or aryl or the heteroforms thereof,
each of l and k is an integer from 0-2 wherein the sum of l and k is 0-3;
Ar is an aryl group substituted with 0-5 noninterfering substituents selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, R₃Si, and NO₂, wherein each R is independently H, alkyl, alkenyl or aryl or the heteroforms thereof, and wherein two of said optional substituents on adjacent positions can be joined to form a fused, optionally substituted aromatic or nonaromatic, saturated or unsaturated ring which contains 3-8 members.

10. The method of claim 6, wherein inhibition of cytokine secretion decreases MM cell replication.

11. A method of potentiating a chemotherapeutic agent for the treatment of multiple myeloma (MM), comprising:
identifying an individual containing one or more MM cells; and
providing a p38 MAP kinase inhibitor to the individual, whereby the one or more MM cells are more sensitive to the chemotherapeutic agent than in the absence of the p p38 MAP kinase inhibitor.

12. The method of claim 10, wherein the chemotherapeutic agent is an apoptosis-promoting agent.

13. The method of claim 11, wherein the chemotherapeutic agent is a proteasome inhibitor.

14. The method of claim 12, wherein the proteasome inhibitor is selected from the group consisting of epoxomicin ((2R)-2-[Acetyl-(N-Methyl-L-Isoleucyl)-L-Isoleucyl-L-Threonyl-L-Leucyl]-2-Methyloxirane); lactacystin (N-Acetyl-L-Cysteine, S-[2R,3S,4R]-3-Hydroxy-2-[(1S)-1-Hydroxy-2-Methylpropyl]-4-Methyl-5-Oxo-2-Pyrolidinecarbonyl]); Z-Ile-Glu(OtBu)-Ala-Leu-H (Carbobenzoxy-L-Isoleucyl-Gamma-t-Butyl-L-Glutamyl-L-Alanyl-L-Leucinal; Z-Leu-Leu-Leu-H [MG 132](Carbobenzoxy-L-Leucyl-L-Leucyl-L-Leucinal); and Z-Leu-Leu-Nva-H [MG 115] (Carbobenzoxy-L-Leucyl-L-Leucyl-L-Norvalinal).

15. The method of claim 12, wherein the proteasome inhibitor is bortezombid (VELCADE), thalidomide or REVIMID.

16. The method of claim 12, wherein the p38 MAPK inhibitor and the proteasome inhibitor are administered simultaneously.

17. The method of claim 11, wherein chemotherapeutic agent is dexamethone.
